(19) United States
(12) Patent Application Publication
(10) Pub. No.: US 2024/0066196 A1
(43) Pub. Date: Feb. 29, 2024

(54) AUTOMATED PERITONEAL DIALYSIS SYSTEM HAVING REUSABLE DRAIN LINE

(71) Applicants: BAXTER INTERNATIONAL INC., Deerfield, IL (US); BAXTER HEALTHCARE SA, Glattpark (Opfikon) (CH)

(72) Inventors: Belur Shanthakumar Shashanka JAIN, Karnataka State, Bangalore (IN); Anoop Thirumattathil ASHOKAN, Bangalore, Karnataka (IN)

(21) Appl. No.: 18/280,027

(22) PCT Filed: Feb. 22, 2022
(86) PCT No.: PCT/US2022/017313

§ 371 Date: Sep. 1, 2023

(30) Foreign Application Priority Data
Mar. 1, 2021 (IN) 202141008530

(51) Int. Cl. A61M 1/14

(52) U.S. Cl. CPC A61M 1/155; A61M 1/1565; A61M 2205/07; A61M 2205/3331

(57) ABSTRACT

A peritoneal dialysis system comprises a cycler including a dialysis fluid pump, a plurality of valve actuators, and a control unit configured to control the dialysis fluid pump and the plurality of valve actuators; and a drain line including a disposable portion, a reusable portion, and a drip chamber located between the disposable portion and the reusable portion, the drip chamber configured to create an air column that dissuades pathogen migration from the reusable portion to the disposable portion. The dialysis fluid pump may include a pneumatic valve manifold and an air pump positioned and arranged to supply pneumatic pressure to the pneumatic valve manifold. The disposable portion of the drain line may be provided as part of a disposable set including a pump actuation portion operable with the dialysis fluid pump, and a valve actuation portion operable with the plurality of valve actuators.

### PRIORITY CLAIM

This application claims priority to and the benefit of Indian Provisional Patent Application No. 202141008530, entitled “Pod Pump Fluid Management System”, filed Mar. 1, 2021, the entire contents of which are incorporated herein by reference and relied upon.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the peritoneal chamber, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities using an automated machine, the automated machine operates typically with a disposable set, which is discarded after a single use. Depending on the complexity of the disposable set, the cost of using a set per day may become significant. Also, daily disposables require space for storage, which can become a nuisance for home owners and businesses. Moreover, daily disposable replacement requires daily setup time and effort by the patient or caregiver at home or at a clinic. There is also a need for APD devices to be portable so that a patient may bring his or her device on vacation or for work travel.

For each of the above reasons, it is desirable to provide a relatively simple, compact APD machine, which operates a simple and cost effective disposable set.

### SUMMARY

The present disclosure sets forth an automated peritoneal dialysis (“APD”) system having a machine or cycler that operates with a disposable set having a pod pump. In one possible configuration for the system, the disposable set includes multiple peritoneal dialysis fluid containers or bags, wherein one of the containers is placed on top of the cycler, which includes a heating plate to heat dialysis fluid located originally in the container as well as dialysis fluid pumped to the container from a second or later container for a subsequent patient fill. In an alternative embodiment, the plate or batch heater is replaced with an inline heater, which heats fresh dialysis fluid as it flows through the patient line to the patient. The disposable pumping pod or pod pump may be oriented vertically as illustrated herein, wherein fluid tubes or lines run horizontally from the pumping pod. An air pump for driving the disposable pod pump and other reusable components herein is located within a housing of the cycler.

The air pump is configured to provide both positive and negative pressure air to the disposable pod pump via a pneumatic valve manifold. The pneumatic valve manifold may include four pneumatic valves, including positive and negative pneumatic valves located between the air pump and the pod pump and reference chamber valves located between the pod pump and a reference chamber (located within the housing), wherein the reference chamber is used for fluid volume determinations discussed herein. In an embodiment, the air pump includes a box-in-a-box noise reducing structure in which inner and out noise reducing or attenuating encloses are provided about a pneumatic pump body to significantly reduce perceptible audible noise outputted by the air pump.

In an embodiment, a first pneumatic pressure sensor is located between the pneumatic valve manifold and the disposable pumping pod (pod pressure sensor). A second pneumatic pressure sensor is located between the pneumatic valve manifold and the reference chamber (reference pressure sensor). The pod and reference pressure sensors are used for the fluid volume determinations discussed herein. The pod pressure sensor is also used to control pumping pressure and to determine an end of stroke for the drawing and discharging of dialysis fluid into and from the pod pump.

The disposable set may include five fluid lines that extend from the disposable pumping pod, including a drain line that extends to a house drain (toilet, sink or bathtub) or to a drain bag. Three peritoneal dialysis fluid containers or bags are provided in one embodiment, one of which sits atop a batch heater as mentioned above. The fifth line is a patient line. The disposable pumping pod mounted vertically to the front or actuation surface of the dialysis cycler, allows the drain line to be located at the top of the pumping pod and the patient line to be located at the bottom of the pumping pod. Such arrangement allows for air in the pod pump to migrate naturally upwardly into the drain line where it can be pumped to drain.

Each of the five fluid lines is fitted into or operates with a pinch valve, which may be an electrically actuated solenoid valve. The pinch valves are failsafe in one embodiment, meaning that upon power loss the valves are biased to close their respective fluid lines. The pinch valves alternatively retain their state upon power loss, but are still part of a failsafe design in cooperation with the pod pump being deactivated upon power loss.

The pinch valves in an embodiment include a rotary nut motor that includes female threads that rotate threadingly around male threads of a threaded shaft. The female threads rotate and cause the threaded shaft to translate back and forth along a central axis extending through the threaded shaft. The rotary nut motor is bidirectional so that the threaded shaft, which carries a spring-loaded plunger in one embodiment, may cause a fluid line to be occluded or opened. A sensor, such as any type of object detecting sensor discussed herein, is provided to look for a proximal end of the threaded shaft protruding through the back of the pinch valve. If the proximal end is detected, indicating a valve open position, at a time when the valve is supposed to be occluding the fluid line, treatment is stopped and an alarm is provided in one embodiment.

The disposable set may include a drain line that is bifurcated into a smaller disposable portion and a longer, distal reusable portion. The disposable and reusable portions are separated by a reusable drip chamber that breaks the effluent drain flow so as to create an air column in a top portion of the drip chamber. The air column prevents any pathogens that develop in the reusable portion of the drain line from traveling through the effluent into the smaller disposable portion of the drain line. The longer, distal reusable portion of the drain line reduces disposable cost and waste.

The drip chamber may include a sensing portion within which the air column is formed and maintained. The sensing portion may include a window or slot for operation with one or more sensor provided by the cycler, e.g., a pair optical sensors. The sensors detect whether air or liquid (effluent) is present. The lower sensor detecting effluent instead of the air column may cause the cycler to take an evasive action, e.g., slow down or stop the present drain flow, while an upper sensor detecting effluent instead of the air column may cause the cycler to take more sever actions, e.g., stop treatment and notify the patient to look for a kink or occlusion in the reusable drain line.

The drip chamber may also be provided with a port for connection to an air pump that pumps into the sensing portion to help maintain the air column or for connection to an air tube having a distal hydrophobic vent that allows air into the sensing portion under negative pressure to help maintain the air column. Positive pressure air may also be used at the end of a patient drain phase to blow out the reusable drain line to prevent or mitigate pathogen growth. To this end, the drip chamber may also be provided with a disinfectant well or sponge to prevent pathogen migration up an inner wall of the sensing portion and for contacting effluent that rises too high in the sensing portion of the drip chamber.

The drip chamber and reusable drain line may be provided with a cycler having the pneumatic pod pumping discussed herein but may alternatively be provided with a cycler having a different type of pumping, such as a durable PD fluid contacting pump that is disinfected after treatment instead of being discarded. In any case, the drip chamber and the reusable portion of the drain line may be capped after treatment when the disposable portion of the drain line has been removed. For example, a distal end of the reusable portion of the drain line may be provided with a tethered cap for capping the distal end, or the cycler may be provided with a blind port for receiving the distal end of the reusable portion of the drain line.

For priming, it is contemplated that the cycler provide a prime sensor/bubble detector, e.g., an optical or capacitive sensor, which operates with the patient line to look for (i) fluid during priming to know that the patient line has been fully primed and (ii) air during treatment. If air is detected during treatment, the air entrained dialysis fluid may be pulled back into the disposable pumping pod and then pumped out to drain.

The pod pump may be constructed in multiple ways. In one embodiment the pod pump includes a rigid, e.g., plastic disposable shell and a flexible sheet, diaphragm or membrane fixed, e.g., ultrasonically welded, to the shell. Here, positive and negative pneumatic pressure is supplied from the air pump and the pneumatic valve manifold to the flexible sheet, diaphragm or membrane. In another embodiment, the pod pump includes two rigid plastic shells, namely, a pneumatic rigid plastic shell and a fluid contacting rigid plastic shell, which are sealed together to hold a flexible sheet, diaphragm or membrane in a sealed manner therebetween. A central pneumatic port is provided in the pneumatic rigid shell, which communicates pneumatically with the air pump and the pneumatic valve manifold. Here, air resides between the rigid plastic shell having the pneumatic port and the flexible membrane. In either of the above embodiments, five fluid ports extend from the fluid contacting rigid plastic shell, which connect to the five fluid lines discussed above. Fresh, fresh heated, or used dialysis fluid resides accordingly between the fluid contacting rigid plastic shell and the flexible membrane. In either embodiment, the flexible membrane is (i) pulled towards the actuation surface under negative pressure to pull fresh or used dialysis fluid into the disposable pumping pod, and (ii) pushed away from the actuation surface under positive pressure to push fresh or used dialysis fluid from the disposable pumping pod.

The pod pump, the flexible plastic sheet, the fluid lines and fluid containers of the disposable set may be made of one or more plastic, e.g., polyvinylchloride (“PVC”) or a non-PVC material, such as polyethylene (“PE”), polyurethane (“PU”) or polycarbonate (“PC”). The housing of the cycler may be made of any of the above plastics, and/or of metal, e.g., stainless steel, steel and/or aluminum. As illustrated herein, the housing of the cycler may take different forms, e.g., the user interface may rotate up or out from the housing or may be integrated with the housing. A lid of the housing may be provided in halves that rotate outwardly to accept portions of a dialysis fluid/heater container or bag. Such arrangement allows an overall size and footprint to be smaller and to not be constrained at least in two dimensions by the size of the fluid/heater container or bag.

A control unit having one or more processor, one or more memory and a video controller operating with a user interface is provided to control each of the fluid valves, each of the pneumatic valves, the air pump, and the heater and to receive signals from each of the pressure sensors, the priming sensor or air detector, and one or more temperature sensor associated with the batch or inline the heater. The user interface may be provided with a touchscreen and/or electromechanical pushbuttons to allow the user or patient to enter parameters for treatment and a display screen for providing information, such as treatment status information. The control unit is also programmed to perform calculations based on the ideal gas law to determine how much fresh or used dialysis fluid has been pumped by the pod pump.

In one embodiment, the control unit is programmed to cause fresh or used dialysis fluid to be drawn into the pod pump using the following procedure. Here, the air pump is configured to be in a negative pressure or suction mode and is placed in pneumatic communication with the disposable pod pump via the opening of the negative pneumatic valve located between the air pump and the pod pump. A desired fluid source valve from which fluid is to be drawn into the disposable pumping pod is opened, e.g., a fresh dialysis fluid source valve, the heater bag valve or the patient valve. The control unit uses pressure feedback from the pod pressure sensor in an algorithm, e.g., a proportional, integral, derivative (“PID”) routine, to regulate the air pump to maintain a desired negative fluid pressure while fluid is pulled into the disposable pumping pod. In an embodiment, the control unit controls current to the air pump to adjust its speed and thus its negative pneumatic pressure output. The desired pressure may be different depending on the fluid source, e.g., −1.5 psig to −3.0 psig for pulling effluent from the patient or higher for pulling fresh PD fluid from a dialysis fluid container.

The control unit in one embodiment uses a second algorithm to sense a spike in negative pressure and/or a corresponding drop in air pump speed to indicate an end of stroke and that the pod pump is filled with fresh or used dialysis fluid, which causes a trigger to stop the air pump.

In one embodiment, the control unit is programmed to measure an initial volume of fresh or used dialysis fluid drawn between the flexible sheet and the fluid contacting rigid plastic shell using two sets of pressure measurements and the ideal gas law. In a first set of pressure measurements, the control unit takes the pressure measurements of (i) the air side of the disposable pumping pod using the pod pressure sensor and (ii) the reference chamber using the reference pressure sensor. After fresh or used dialysis fluid is drawn into the pod pump, the control unit in a second set of pressure measurements opens one or more pneumatic valve(s) to allow the air side of the disposable pumping pod and the reference chamber to communicate pneumatically. Here, both the pod and reference pressure sensors measure the pressure of the combined cavity. Then, with all values on the right side of the following equation known or measured (the volume of the reference chamber is known), the control unit calculates the volume of fluid pulled into the disposable pumping pod is as follows:

V_{fluid initial}=V_{reference chamber}*(P_{ref final}−P_{ref initial})/(P_{pump initial}−P_{pump final})

In one embodiment, the control unit is programmed to cause fresh or used dialysis fluid to be pumped from the pod pump using the following procedure. Here, the air pump is configured to be in a positive pressure mode, which is placed in pneumatic communication with the disposable pumping pod via the opening of the positive pneumatic valve located between the air pump and the pod pump. A desired fluid destination valve through which fluid is to be delivered from the disposable pumping pod is opened, e.g., the heater bag valve, the patient valve or the drain valve. The control unit uses pressure feedback from the pod pressure sensor in the pressure algorithm, e.g., PID routine, to regulate the air pump to maintain a desired positive fluid pressure while fluid is discharged from the disposable pumping pod. Again, the control unit controls current to the air pump to adjust its speed and thus its positive pneumatic pressure output. The desired pressure may be different depending on the fluid destination, e.g., 3.0 psig to 8.0 psig for pushing fresh, heated dialysis fluid to the patient or higher for pushing to drain or the heating container.

The control unit in one embodiment uses an additional algorithm to sense a spike in positive pressure and/or a corresponding drop in air pump speed to indicate an end of stroke and that the pod pump has been emptied of fresh or used dialysis fluid, which causes a trigger to stop the air pump.

In one embodiment, the control unit is programmed to measure a final volume of fresh or used dialysis fluid located between the flexible sheet and the fluid contacting rigid plastic shell using the same two sets of pressure measurements and the ideal gas law. In a first set of pressure measurements, the control unit takes the pressure measurements of (i) the air side of the disposable pumping pod using the pod pressure sensor and (ii) the reference chamber using the reference pressure sensor. After fresh or used dialysis fluid is pumped from the pod pump, the control unit in a second set of pressure measurements opens one or more pneumatic valve(s) to allow the air side of the disposable pumping pod and the reference chamber to communicate pneumatically. Here, both the pod and reference pressure sensors measure the pressure of the combined cavity. Then, with all values on the right side of the following equation known or measured, the control unit calculates the volume of fluid remaining in the pumping pod after the discharge stroke as follows:

V_{fluid final}=V_{reference chamber}*(P_{ref final}−P_{ref initial})/(P_{pump initial}−P_{pump final})

The control unit then calculates the volume of fluid pumped from the pod pump by calculating the difference between the calculated pumping chamber volume before (V_{fluid initial}) and after (V_{fluid final}) pumping. The above steps or procedures are repeated until a required or prescribed volume of fresh or used dialysis fluid is pumped. The same pumping regime just described is used to pump fluid from any fluid source to any destination.

In light of the disclosure set forth herein, and without limiting the disclosure in any way, in a first aspect of the present disclosure, which may be combined with any other aspect, or portion thereof, a peritoneal dialysis system comprises a cycler including a pneumatic valve manifold, an air pump positioned and arranged to supply pneumatic pressure to the pneumatic valve manifold without intervening pneumatic storage, a pneumatic pressure sensor positioned and arranged to detect pneumatic pressure, and a control unit configured to use an output of the pneumatic pressure sensor as feedback to adjust the air pump according to a set pneumatic pressure; and a disposable set including a pod pump having a flexible sheet, one side of the flexible sheet positioned and arranged during operation to receive pneumatic pressure via the air pump and pneumatic valve manifold.

In a second aspect of the present disclosure, which may be combined with any other aspect, or portion thereof, the air pump is configured to supply positive and negative pneumatic pressure.

In a third aspect of the present disclosure, which may be combined with any other aspect, or portion thereof, the pneumatic valve manifold includes a positive pneumatic valve positioned between the air pump and the side of the flexible sheet positioned and arranged during operation to receive pneumatic pressure and a negative pneumatic valve positioned between the air pump and the side of the flexible sheet positioned and arranged during operation to receive pneumatic pressure, and wherein the control unit is configured to open the positive pneumatic valve when the air pump is supplying positive pneumatic pressure and to open the negative pneumatic valve when the air pump is supplying negative pneumatic pressure.

In a fourth aspect of the present disclosure, which may be combined with any other aspect, or portion thereof, the pneumatic valve manifold further includes at least one additional pneumatic valve positioned and arranged to allow a reference chamber to communicate pneumatically with the side of the flexible sheet positioned and arranged during operation to receive pneumatic pressure.

In a fifth aspect of the present disclosure, which may be combined with any other aspect, or portion thereof, the pressure sensor is a first pressure sensor positioned and is arranged between the pneumatic valve manifold and the side of the flexible sheet positioned and arranged during operation to receive pneumatic pressure, and wherein the cycler also includes a second pressure sensor positioned and arranged between the pneumatic valve manifold and the reference chamber.

In a sixth aspect of the present disclosure, which may be combined with any other aspect, or portion thereof, the control unit is further configured to use outputs from the first and second pressure sensors in combination with a sequence of the positive pneumatic valve, negative pneumatic valve and the at least one additional pneumatic valve and an ideal gas law equation to compute at least one of (i) an initial dialysis fluid volume in the pod pump after fresh or used dialysis fluid is drawn into the pod pump or (ii) a final dialysis fluid volume in the pod pump after fresh or used dialysis fluid is pumped from the pod pump.

In a seventh aspect of the present disclosure, which may be combined with any other aspect, or portion thereof, the control unit is further configured to subtract (ii) from (i) to determine a volume of the fresh or used dialysis fluid pumped from the pod pump.

In an eighth aspect of the present disclosure, which may be combined with any other aspect, or portion thereof, the control unit is further configured to use at least one of a sensed speed of the air pump or the output of the pneumatic pressure sensor to determine that the flexible sheet has completed (i) drawing fresh or used dialysis fluid into the pod pump or (ii) pumping fresh or used dialysis fluid from the pod pump.

In a ninth aspect of the present disclosure, which may be combined with any other aspect, or portion thereof, the cycler further includes at least one of (i) a batch or inline dialysis fluid heater under control of the control unit or (ii) an air or priming sensor outputting to the control unit.

In a tenth aspect of the present disclosure, which may be combined with any other aspect, or portion thereof, the cycler further includes an actuation surface, and wherein the one side of the flexible sheet is positioned and arranged during operation to receive pneumatic pressure through the actuation surface via the air pump and pneumatic valve manifold.

In an eleventh aspect of the present disclosure, which may be combined with any other aspect, or portion thereof, the disposable cassette includes a plurality of dialysis fluid lines extending from the pod pump, wherein the cycler includes a plurality of fluid valves provided along the actuation surface for interfacing with the plurality of dialysis fluid lines, and wherein the fluid valves are optionally spring actuated to occlude the plurality of dialysis fluid lines.

In a twelfth aspect of the present disclosure, which may be combined with any other aspect, or portion thereof, the disposable cassette includes a fluid contacting rigid plastic shell, wherein the flexible sheet is fixed to the fluid contacting rigid plastic shell.

In a thirteenth aspect of the present disclosure, which may be combined with any other aspect, or portion thereof, the disposable cassette includes a pneumatic rigid plastic shell and a fluid contacting rigid plastic shell, which are sealed together to hold the flexible sheet therebetween.

In a fourteenth aspect of the present disclosure, which may be combined with any other aspect, or portion thereof, the air pump is provided within a noise-reducing enclosure.

In a fifteenth aspect of the present disclosure, which may be combined with any other aspect, or portion thereof, a peritoneal dialysis system comprises a disposable set including a pod pump having a flexible sheet, one side of the flexible sheet positioned and arranged during operation to receive pneumatic pressure; and a cycler including a pneumatic valve manifold for providing pneumatic pressure to the side of the flexible sheet positioned and arranged during operation to receive pneumatic pressure, an air pump positioned and arranged to supply pneumatic pressure to the pneumatic valve manifold without intervening pneumatic storage, a reference chamber in pneumatic communication with the pneumatic valve manifold, a first pneumatic pressure sensor positioned and arranged to detect pneumatic pressure outputted by the air pump, a second pressure sensor positioned and arranged to detect pneumatic pressure within the reference chamber, and a control unit configured to use outputs from the first and second pressure sensors in combination with a sequence of the pneumatic valve manifold and an ideal gas law equation to compute at least one of (i) an initial dialysis fluid volume in the pod pump after fresh or used dialysis fluid is drawn into the pod pump or (ii) a final dialysis fluid volume in the pod pump after fresh or used dialysis fluid is pumped from the pod pump.

In a sixteenth aspect of the present disclosure, which may be combined with any other aspect, or portion thereof, the control unit is further configured to subtract (ii) from (i) to determine a volume of the fresh or used dialysis fluid pumped from the pod pump.

In a seventeenth aspect of the present disclosure, which may be combined with any other aspect, or portion thereof, the control unit is further configured to repeat (i) and (ii) and to subtract (ii) from (i) until a prescribed volume of fresh or used dialysis fluid is pumped.

In an eighteenth aspect of the present disclosure, which may be combined with any other aspect, or portion thereof, the pneumatic valve manifold includes a positive pneumatic valve positioned between the air pump and the side of the flexible sheet positioned and arranged during operation to receive pneumatic pressure and a negative pneumatic valve positioned between the air pump and the side of the flexible sheet positioned and arranged during operation to receive pneumatic pressure, and at least one additional pneumatic valve positioned and arranged to allow the reference chamber to communicate pneumatically with the side of the flexible sheet positioned and arranged during operation to receive pneumatic pressure, and wherein the control unit is configured to use outputs from the first and second pressure sensors prior to and after the at least one additional pneumatic valve is opened.

In a nineteenth aspect of the present disclosure, which may be combined with any other aspect, or portion thereof, the sequence of the pneumatic valve manifold includes opening the negative pneumatic valve to draw fresh or used dialysis fluid into the pod pump prior to opening the at least one additional pneumatic valve.

In a twentieth aspect of the present disclosure, which may be combined with any other aspect, or portion thereof, the sequence of the pneumatic valve manifold includes opening the positive pneumatic valve to pump fresh or used dialysis fluid from the pod pump prior to opening the at least one additional pneumatic valve.

In a twenty-first second aspect of the present disclosure, which may be combined with any other aspect, or portion thereof, the control unit is configured to open the positive pneumatic valve when the air pump is supplying positive pneumatic pressure and to open the negative pneumatic valve when the air pump is supplying negative pneumatic pressure.

In a twenty-second second aspect of the present disclosure, which may be combined with any other aspect, or portion thereof, a peritoneal dialysis system comprises a cycler including a dialysis fluid pump, a plurality of valve actuators, and a control unit configured to control the dialysis fluid pump and the plurality of valve actuators; and a disposable set including a pump actuation portion operable with the dialysis fluid pump, a valve actuation portion operable with the plurality of valve actuators and a drain line, the drain line including a disposable portion, a reusable portion, and a drip chamber located between the disposable portion and the reusable portion, the drip chamber configured to create an air column that dissuades pathogen migration from the reusable portion to the disposable portion.

In a twenty-third aspect of the present disclosure, which may be combined with any other aspect, or portion thereof, the drip chamber is reusable and provided with the reusable portion of the drain line.

In a twenty-fourth second aspect of the present disclosure, which may be combined with any other aspect, or portion thereof, the dialysis fluid pump includes a pneumatic valve manifold and an air pump positioned and arranged to supply pneumatic pressure to the pneumatic valve manifold.

In a twenty-fifth aspect of the present disclosure, which may be combined with any other aspect, or portion thereof, the pump actuation portion of the disposable set includes a pod pump having a flexible sheet, one side of the flexible sheet positioned and arranged during operation to receive pneumatic pressure via the air pump and pneumatic valve manifold.

In a twenty-sixth aspect of the present disclosure, which may be combined with any other aspect, or portion thereof, the plurality of valve actuators include pinch valve actuators.

In a twenty-seventh second aspect of the present disclosure, which may be combined with any other aspect, or portion thereof, the valve actuation portion of the disposable set includes a plurality of lines, including the drain line, positioned and arranged to be actuated by the pinch valve actuators

In a twenty-eighth aspect of the present disclosure, which may be combined with any other aspect, or portion thereof, the peritoneal dialysis system includes a pneumatic pressure sensor positioned and arranged to detect pneumatic pressure, and a control unit configured to use an output of the pneumatic pressure sensor as feedback to adjust the air pump according to a set pneumatic pressure.

In a twenty-ninth aspect of the present disclosure, which may be combined with any other aspect, or portion thereof, the reusable portion of the drain line is longer than the disposable portion of the drain line.

In a thirtieth aspect of the present disclosure, which may be combined with any other aspect, or portion thereof, the peritoneal dialysis system includes a connector located at an end of the disposable portion of the drain line, the connector configured to connect to the drip chamber, the connector configured to dissuade touch contamination.

In a thirty-first aspect of the present disclosure, which may be combined with any other aspect, or portion thereof, the cycler includes a clip for holding the drip chamber.

In a thirty-second aspect of the present disclosure, which may be combined with any other aspect, or portion thereof, the cycler includes a sensor outputting to the control unit, the drip chamber positioned and arranged to enable the sensor to sense the air column.

In a thirty-third aspect of the present disclosure, which may be combined with any other aspect, or portion thereof, the sensor is an optical sensor, an ultrasonic sensor, a capacitance sensor, a proximity sensor, a magnetic sensor or combinations thereof.

In a thirty-fourth aspect of the present disclosure, which may be combined with any other aspect, or portion thereof, the sensor incudes first and second sensors spaced elevationally apart, the control unit programmed to react differently to the first sensor versus the second sensor detecting an interruption of the air column.

In a thirty-fifth aspect of the present disclosure, which may be combined with any other aspect, or portion thereof, the drip chamber includes a window or slot for operation with the sensor.

In a thirty-sixth aspect of the present disclosure, which may be combined with any other aspect, or portion thereof, the drip chamber includes a well configured to receive and hold disinfectant.

In a thirty-seventh aspect of the present disclosure, which may be combined with any other aspect, or portion thereof, the peritoneal dialysis system includes a disinfectant reservoir positioned and arranged to deliver disinfectant to the well.

In a thirty-eighth aspect of the present disclosure, which may be combined with any other aspect, or portion thereof, the peritoneal dialysis system includes a disinfectant sponge configured for insertion into the drip chamber.

In a thirty-ninth aspect of the present disclosure, which may be combined with any other aspect, or portion thereof, the disinfectant sponge is provided as part of the disposable portion of the drain line.

In a fortieth first aspect of the present disclosure, which may be combined with any other aspect, or portion thereof, the drip chamber includes a port connectable to a pneumatic line for receiving air to help maintain the air column.

In a forty-first aspect of the present disclosure, which may be combined with any other aspect, or portion thereof, the peritoneal dialysis system includes an air pump for delivering air through the pneumatic line or a hydrophobic vent placed at a distal end of the pneumatic line enabling air to be drawn into the drip chamber under negative pressure.

In a forty-second aspect of the present disclosure, which may be combined with any other aspect, or portion thereof, a pinch valve includes a linear actuator; a shaft driven be the linear actuator; a plunger moved by the shaft; a housing defining an aperture through which the plunger extends to occlude a fluid line; and a sensor positioned and arranged relative to the linear actuator so as to sense a portion of the shaft indicating a valve open position.

In a forty-third aspect of the present disclosure, which may be combined with any other aspect, or portion thereof, the housing is fixed to the linear actuator.

In a forty-fourth aspect of the present disclosure, which may be combined with any other aspect, or portion thereof, the shaft is a threaded shaft and the linear actuator includes rotary nut motor threadingly engaged to the threaded shaft for translating the threaded shaft in a first direction when the rotary nut motor rotates in a first direction and translating the threaded shaft in a second direction when the rotary nut motor rotates in a second direction.

In a forty-fifth aspect of the present disclosure, which may be combined with any other aspect, or portion thereof, the pinch valve includes a collar coupled to the shaft, a mounting flange fixed to the plunger, and a spring positioned between the plunger and the collar, the spring biased to pull the mounting flange against the collar when the plunger does not engage the fluid line, the spring configured to be compressed when the plunger engages the fluid line.

In a forty-sixth aspect of the present disclosure, which may be combined with any other aspect, or portion thereof, the sensor is mounted to the linear actuator.

In a forty-seventh aspect of the present disclosure, which may be combined with any other aspect, or portion thereof, the sensor is positioned and arranged to sense a proximal end of the shaft extending though a back of the linear actuator.

In a forty-eighth aspect of the present disclosure, which may be combined with any other aspect, or portion thereof, the sensor is an optical, capacitive, or ultrasonic sensor.

In a forty-ninth aspect of the present disclosure, which may be combined with any other aspect, or portion thereof, the peritoneal dialysis system a sealing mechanism for sealing a distal end of the disposable portion of the drain line after treatment.

In a fiftieth aspect of the present disclosure, which may be combined with any other aspect, or portion thereof, the sealing mechanism includes a tethered cap or a blind port provided at the cycler.

In a fifty-first aspect of the present disclosure, which may be combined with any other aspect, or portion thereof, the peritoneal dialysis system (i) includes a sealing mechanism for sealing the drip chamber after treatment or (ii) is configured such that the disposable portion of the drain line remains connected to the drip chamber after treatment.

In a fifty-second aspect of the present disclosure, which may be combined with any other aspect, or portion thereof, any of the features, functionality and alternatives described in connection with any one or more of FIGS. 1A to 13 may be combined with any of the features, functionality and alternatives described in connection with any other of FIGS. 1A to 13.

In light of the above aspects and present disclosure, it is an advantage of the present disclosure to provide a relatively volumetrically accurate automated peritoneal dialysis (“APD”) cycler.

It is another advantage of the present disclosure to provide an APD cycler that achieves relatively precise pressure control.

It is a further advantage of the present disclosure to provide a relatively quiet APD cycler.

It is still another advantage of the present disclosure to provide a pneumatically operated APD cycler that does not require pressure storage devices.

It is still a further advantage of the present disclosure to provide an APD system that is able to build motive fluid or pneumatic pressure in a relatively simple manner.

It is yet another advantage of the present disclosure to provide an APD system that employs a relatively low cost disposable set.

It is yet a further advantage of the present disclosure to provide an APD system that employs a primarily reusable drain line, which saves disposable cost.

Still another advantage of the present disclosure is to provide an APD system that employs improved pinch valves.

Still a further advantage of the present disclosure is to provide an APD system that employs a reduced noise air pump.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A is a perspective view of one embodiment of an automated peritoneal dialysis (“APD”) cycler using a pod pump of the system of the present disclosure.

FIG. 1B is a top sectioned view of one embodiment of an APD cycler using a pod pump of the present disclosure.

FIG. 2 is a schematic view of one embodiment of an APD cycler using a pod pump of the present disclosure.

FIG. 3 is an exploded perspective view of one embodiment for a disposable pod pump of the present disclosure.

FIG. 4 is an exploded perspective view of another embodiment for a disposable pod pump of the present disclosure.

FIG. 5 is a process flow diagram illustrating one embodiment for determining fresh or used dialysis fluid volume delivery.

FIG. 6 is a perspective view of an alternative embodiment for an APD cycler using a pod pump of the system of the present disclosure.

FIGS. 7A and 7B are sectioned perspective views of an actuation surface portion of the APD cycler of FIG. 6 showing the disposable set unloaded and loaded, respectively.

FIGS. 8A to 10 are perspective and sectioned elevation views of one embodiment for a drip chamber allowing for the reuse of a large portion of the drain line.

FIG. 11 is a perspective view of a further alternative embodiment for an APD cycler using a pod pump of the system of the present disclosure.

FIG. 12 is an exploded perspective view of one embodiment for a pinch valve useable with any version of the APD cycler and associated system of the present disclosure.

FIG. 13 is a front elevation view of one embodiment for a reduced noise air pump useable with any version of the APD cycler and associated system of the present disclosure.

### DETAILED DESCRIPTION

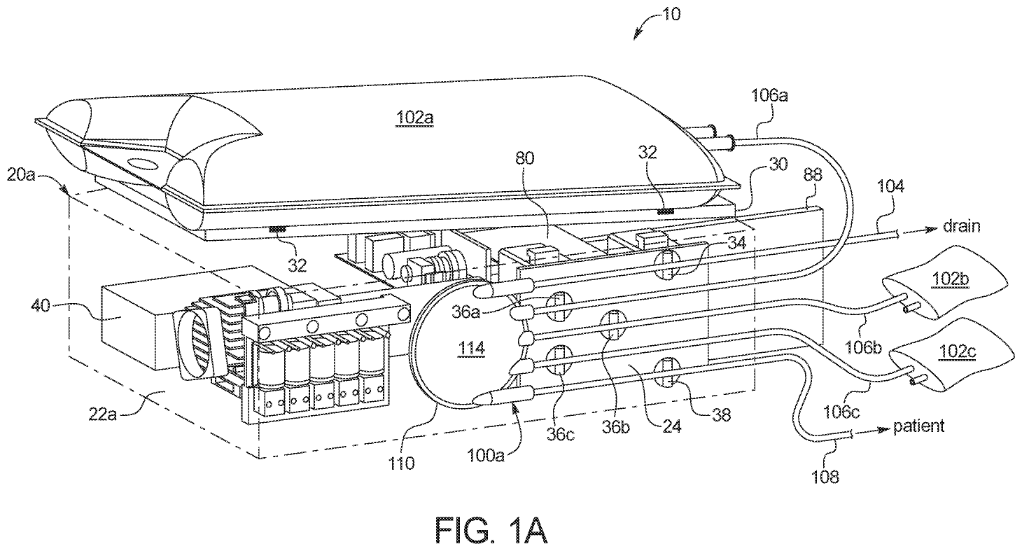

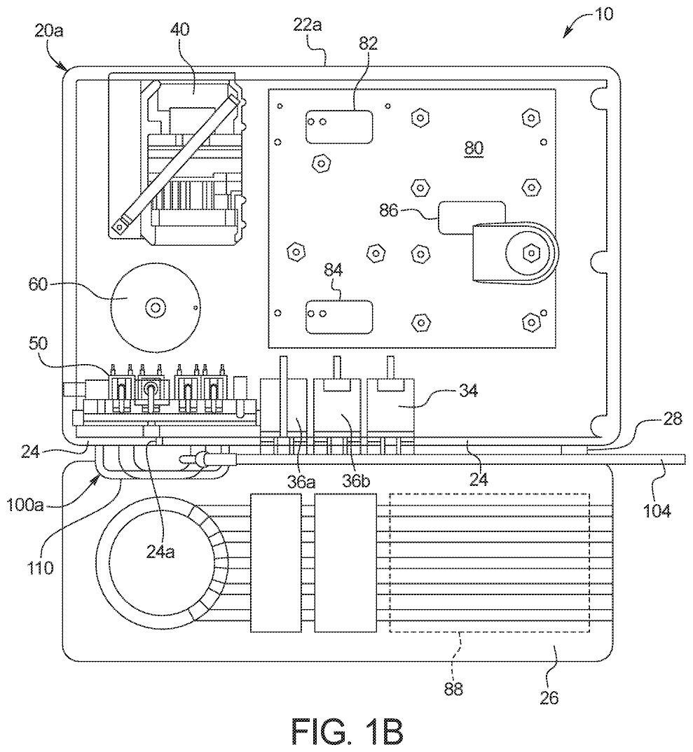

Referring now to the drawings and in particular to FIGS. 1A and 1B, an embodiment of system 10 includes an automated peritoneal dialysis (“APD”) cycler 20a having a housing 22a, which operates with a disposable set 100a that organizes tubing and performs many functions discussed herein. Disposable set 100a includes a pod pump 110, which in one embodiment is covered on one side by a flexible plastic sheet 112 that is easy and cost effective to manufacture, e.g., so that two rigid plastic pieces do not have to be welded together.

As illustrated in FIGS. 1A and 1B, disposable set 100a including pod pump 110 is inserted for operation inside of APD cycler 20a, for example, in between an actuation surface 24 and a door 26 of the APD cycler. Door 26, for example, hinges open via one or more hinge 28 located along a bottom of cycler housing 22a, adjacent to actuation surface 24. In the illustrated implementation, pod pump 110 of disposable set 100a is mounted so that plastic sheet 112 of pod pump 110 when mounted for operation is located facing actuation surface 24 of cycler 20a. Door 26 when closed constrains pod pump 110 against actuation surface 24 during operation in one embodiment.

In one possible configuration for system 10 as illustrated in FIGS. 1A and 1B, disposable set 100a includes multiple peritoneal dialysis fluid containers or bags 102a to 102c, wherein one of the containers, e.g., container 102a, is placed on top of cycler 20a, which includes a dialysis fluid heater 30 to heat dialysis fluid located originally in container 102a as well as dialysis fluid pumped to container 102a from second and third containers 102b and 102c for subsequent patient fills. In an alternative embodiment, plate or batch heater 30 is replaced with an inline dialysis fluid heater (not illustrated), which heats fresh dialysis fluid as it flows through a patient line to the patient. A serpentine inline heating pathway may be provided along the patient line to aid the inline heating. The serpentine inline heating pathway may be placed on top of cycler 20a or be inserted into the cycler for heating, e.g., via resistive plate heating.

Disposable pumping pod or pod pump 110 is oriented vertically in the illustrated embodiment of FIGS. 1A and 1B, wherein fluid tubes or lines run horizontally from the pumping pod. In the illustrated embodiment, pod pump 110 includes a fluid contacting fluid contacting rigid plastic shell 114 to which flexible sheet 112 is attached, e.g., ultrasonically sealed. FIGS. 1A, 3 and 4 illustrate that fluid contacting rigid plastic shell 114 includes or defines a drain port 114a that accepts a drain line 104 in a sealed manner, wherein drain line 104 extends to a drain container or house drain, e.g., toilet, bathtub or sink (not illustrated). Fluid contacting rigid plastic shell 114 includes or defines a first dialysis fluid port 116a that accepts a first dialysis fluid line 106a in a sealed manner, wherein first dialysis fluid line 106a extends to fresh dialysis fluid container 102a positioned on the top of cycler 20a, which doubles as the fluid heating container. Fluid contacting rigid plastic shell 114 includes or defines a second dialysis fluid port 116b that accepts a second dialysis fluid line 106b in a sealed manner, wherein second dialysis fluid line 106b extends to fresh dialysis fluid container 102b. Fluid contacting rigid plastic shell 114 includes or defines a third dialysis fluid port 116c that accepts a third dialysis fluid line 106c in a sealed manner, wherein third dialysis fluid line 106c extends to fresh dialysis fluid container 102c. Fluid contacting rigid plastic shell 114 includes or defines a patient port 118 that accepts a patient line 108 in a sealed manner, wherein patient line 108 extends to a patient connector (not illustrated) that connects to a patient's transfer set and communicates fluidly with the patient's indwelling peritoneal dialysis catheter.

Disposable set 100a may alternatively provide more or less than the illustrated five fluid lines, e.g., the number of fresh dialysis fluid containers may be varied. Fresh dialysis fluid containers 102a to 102c may contain the same or different types and volumes of fresh dialysis fluids. For example, fresh dialysis fluid containers 102a to 102c may contain different levels of dextrose or glucose. One of the containers may contain a different formulation of fresh dialysis fluid, e.g., icodextrin, for a patient's last fill.

With disposable set 100a and pumping pod 110 mounted vertically to actuation surface 24 of cycler 20a, drain line 104 is located at the top of the pumping pod, while patient line 18 is located at the bottom of the pumping pod. Such arrangement allows for air in pod pump 110 to migrate naturally upwardly into drain line 104 where it can be pumped to the house drain or drain container.

To load disposable set 100a and pod pump 110, the patient or user in one embodiment opens door 26 as illustrated in FIG. 1B and loads disposable set 100a into the inside of the door as the door lays flat or almost flat on a table, nightstand, desk, etc. The patient or caregiver then closes door 26, causing pod pump 110 and fluid lines 104, 106a to 106c and 108 to come into registry, respectively, with one or more pneumatic delivery aperture or port 24a (FIG. 1B) formed in actuation surface 24 and with pinch valves 34, 36a to 36c and 38 located along actuation surface 24. The patient or caregiver then loads fresh dialysis fluid container 102a onto resistive plate or batch dialysis fluid heater 30 assuming that batch heating is provided. Dialysis fluid containers or bags 102b and 102c may be located in a convenient position on the table, nightstand, desk, etc.

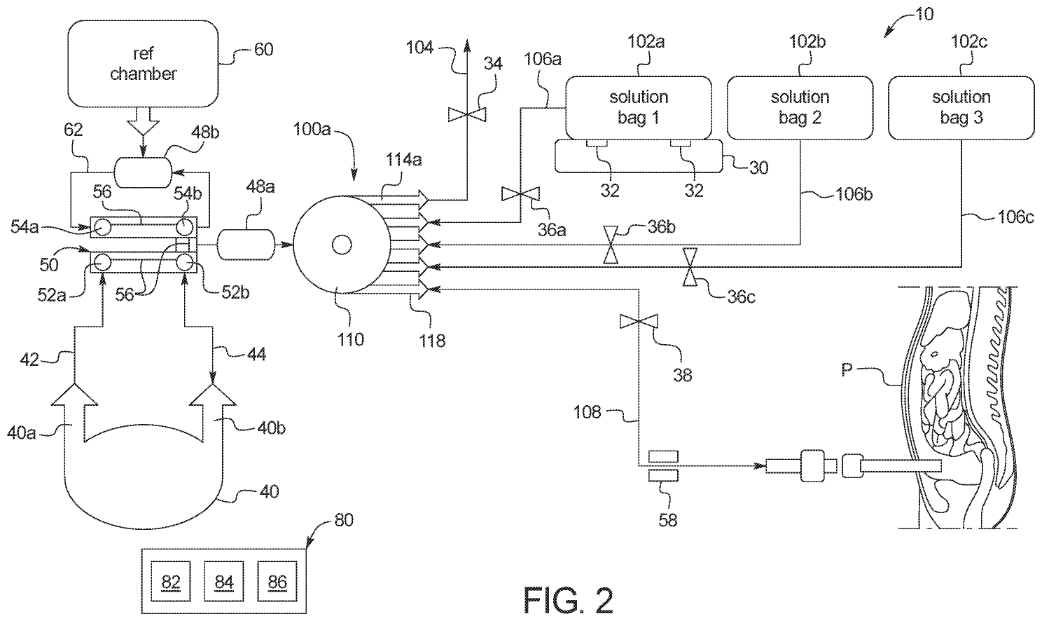

Referring additionally to FIG. 2, disposable set 100a, dialysis fluid heater 30 and pinch valves 34, 36a to 36c and 38 just described are illustrated again along with other components of system 10. Dialysis fluid heater 30, priming/air sensor 58, and pinch valves 34, 36a to 36c and 38 and each of the components to the left of pod pump 110 in FIG. 2 are durable, reusable components. Cycler 20a provides an air pump 40 for pneumatically driving disposable pod pump 110. Air pump 40 in one embodiment is bidirectional and can supply positive and negative pneumatic pressure, e.g., positive pneumatic pressure out of positive port 40a and negative pneumatic pressure out of negative port 40b. Suitable air pumps 40 may for example be diaphragm pumps, scroll pumps or piston pumps. Air pump 40 may be electrically actuated such that the amount of current delivered to the air pump determines the speed of the pump and the pressure and flowrate of air delivered to pod pump 110.

In the illustrated embodiment, air pump 40 delivers both positive and negative pressure air to disposable pod pump 110 via a pneumatic valve manifold 50. In particular, positive pressure line 42 leads from positive port 40a to pneumatic valve manifold 50, while negative pressure line 44 leads from negative port 40b to pneumatic valve manifold 50. Pneumatic valve manifold 50 in the illustrated embodiment includes four pneumatic valves, including positive and negative pneumatic valves 52a and 52b located between air pump 40 and pod pump 110, and reference chamber valves 54a and 54b located between pod pump 110 and a known volume reference chamber 60, which is used for fluid volume determinations discussed herein. Pneumatic valves 52a, 52b, 54a and 54b in an embodiment are electrically actuated solenoid valves that open upon being energized so as to operate in a failsafe way. Pneumatic valve manifold 50 also includes pneumatic lines 56 linking positive and negative pneumatic valves 52a and 52b and reference chamber valves 54a and 54b. In an embodiment, one of reference chamber valves 54a and 54b pneumatically connects reference chamber 60 to pneumatic rigid plastic shell 120 when needed, while the other of reference chamber valves 54a and 54b pneumatically connects reference chamber 60 to ambient for venting the reference chamber when needed.

In the illustrated embodiment, a first pneumatic pressure sensor 48a is located between pneumatic valve manifold 50 and disposable pumping pod 110 (pod pressure sensor). A second pneumatic pressure sensor 48b is located between pneumatic valve manifold 50 and reference chamber 60 (reference pressure sensor). Pod and reference pressure sensors 48a and 48b are used for the volume determinations discussed herein. Pod pressure sensor 48a is also used to control the pumping pressure of pod pump 110 and to determine an end of stroke for the drawing and discharging of fresh and used dialysis fluid into and from the pod pump. Pod pressure sensor 48a enables system 10 to not provide a pressure sensor located along patient line 108, however, such a patient line pressure sensor may be provided if desired, e.g., for redundancy. Reference pressure sensor 48b is located along a pneumatic loop 62 pneumatically linking pneumatic valve manifold 50 to reference chamber 60.

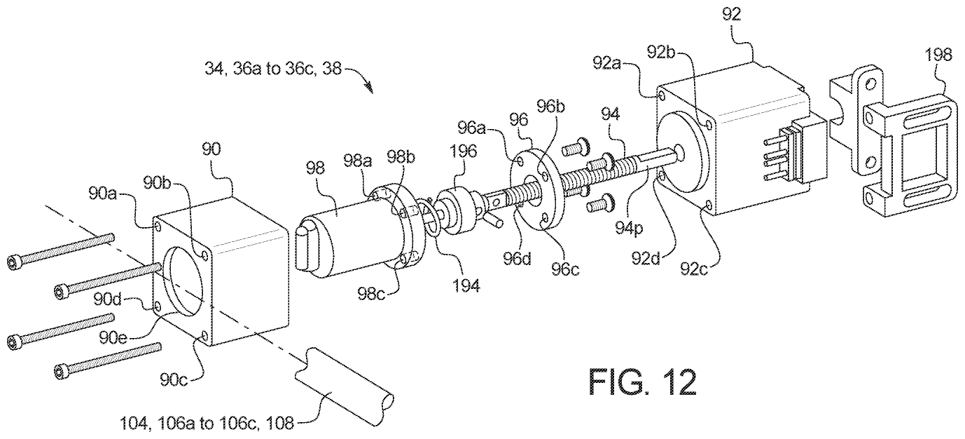

FIG. 2 also illustrates that each of the five fluid lines 104, 106a to 106c and 108 is fitted into or operates respectively with a pinch valve 34, 36a to 36c and 38, which may each be an electrically actuated solenoid valve. Pinch valves 34, 36a to 36c and 38 are failsafe in one embodiment, meaning that upon power loss the valves are biased to close their respective fluid lines. The pinch valves alternatively retain their state upon power loss, but are still part of a failsafe design in cooperation with pod pump 110 being deactivated upon power loss. A suitable valve for pinch valves 34, 36a to 36c and 38 is illustrated and described in connection with FIG. 12.

As illustrated in FIG. 2, to draw fresh dialysis fluid into pod pump 110, negative pressure is applied via air pump 40 and one of dialysis fluid pinch valves 36a to 36c is opened, while all other pinch valves are closed. To draw fresh, heated dialysis fluid into pod pump 110, negative pressure is applied and dialysis fluid/heater pinch valve 36a is opened, while all other pinch valves are closed. To draw used dialysis fluid from patient P into pod pump 110, negative pressure is applied and patient valve 38 is opened, while all other pinch valves are closed. To push fresh, heated dialysis fluid from pod pump 110 to patient P, positive pressure is applied and patient valve 38 is opened, while all other pinch valves are closed. To push fresh, unheated dialysis fluid from pod pump 110 to be heated, positive pressure is applied via air pump 40 and dialysis fluid pinch valve 36a is opened, while all other pinch valves are closed. To push used dialysis fluid from pod pump 110 to drain, positive pressure is applied and drain pinch valve 34 is opened, while all other pinch valves are closed.

For priming, it is contemplated that cycler 20a provide a prime sensor/bubble detector 58, e.g., an optical or capacitive sensor, which operates with patient line 108 to look for (i) fresh dialysis fluid during priming to know that patient line 108 has been fully primed and (ii) air in patient line 108 during treatment. If air is detected during treatment, the air entrained dialysis fluid may be pulled back along patient line 108 into disposable pumping pod 110, and then pumped out to drain via drain line 104. Alternatively, it is contemplated to allow a certain amount of air to build within pod pump 110, e.g., at the top of the fluid contacting shell 114, before being delivered to drain.

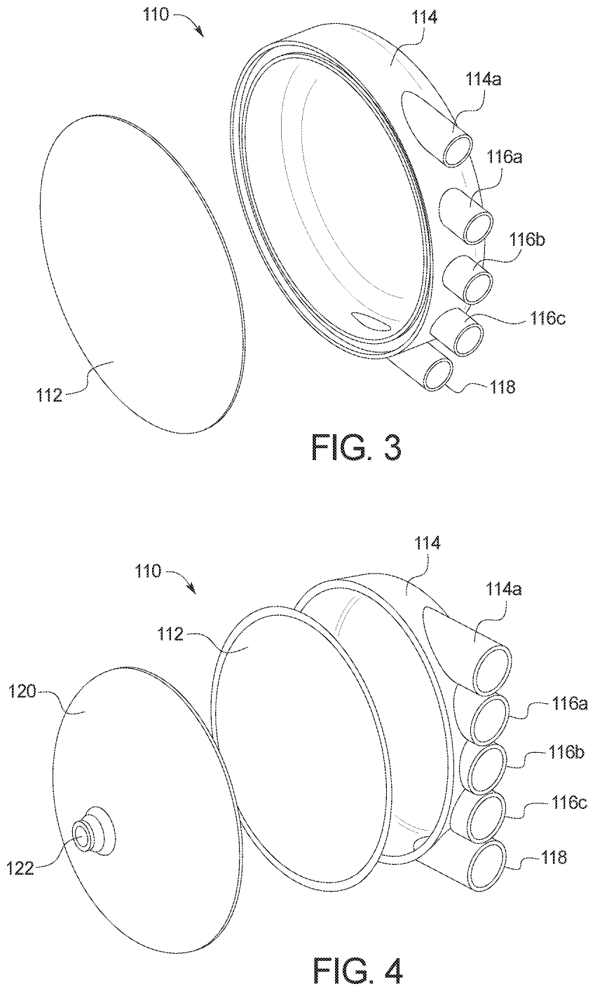

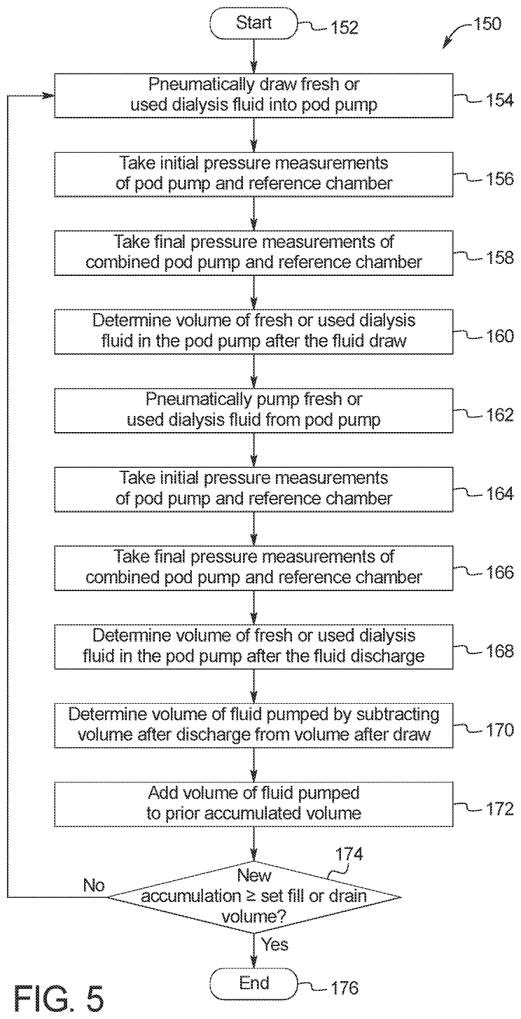

FIGS. 3 and 4 illustrate that pod pump 110 may be constructed in multiple ways. In FIG. 3, pod pump 110 includes rigid, e.g., disposable fluid contacting shell 114 and flexible sheet, diaphragm or membrane 112 fixed, e.g., ultrasonically welded, heat sealed or adhered to shell 114. Here, positive and negative pneumatic pressure is supplied from air pump 110, pneumatic valve manifold 50 and pneumatic delivery aperture or port 24a formed in actuation surface 24 to flexible sheet, diaphragm or membrane 112. Notably, pneumatic storage tanks or bottles of stored positive and negative pneumatic pressure are not needed. Pressurized air is supplied directly from air pump 40 to pod pump 110 via the pneumatic lines and valves. Plastic disposable shell 114 in FIG. 3 includes drain port 114a, dialysis fluid ports 116a to 116c and patient port 118 described above. Plastic sheet 112 may be pulled pneumatically into a cavity formed in actuation surface 24 and having a same or similar diameter as disposable shell 114 to draw fresh or used dialysis fluid into the fluid contacting shell 114. Plastic sheet 112 is pushed pneumatically into fluid contacting shell 114 to dispel or discharge fresh or used dialysis fluid from pod pump 110.

FIG. 4 illustrates an alternative embodiment for pod pump 110, which includes two rigid plastic shells, namely, fluid contacting rigid plastic shell 114 and an additional pneumatic rigid plastic shell 120, which are sealed together to hold flexible sheet, diaphragm or membrane 112 in a sealed manner therebetween. A central port 122 is provided in pneumatic rigid plastic shell 120, which is a pneumatic port that communicates pneumatically with air pump 40 and pneumatic valve manifold 50, e.g., seals to or within the pneumatic aperture 24a provided in actuation surface 24. Here, air and pneumatic pressure resides between pneumatic shell 120 and flexible membrane 112. Plastic disposable shell 114 in FIG. 4 again includes drain port 114a, dialysis fluid ports 116a to 116c and patient port 118 described above. In an embodiment, flexible sheet 112 is attached, e.g., ultrasonically welded, heat sealed or adhered to one of rigid shells 114 or 120, after which that subassembly is attached, e.g., ultrasonically welded, heat sealed or adhered to the other one of rigid shells 114 or 120. In the embodiment of FIG. 4, plastic sheet 112 is pulled pneumatically into pneumatic rigid plastic shell 120 to draw fresh or used dialysis fluid into the fluid contacting shell 114. Plastic sheet 112 is again pushed pneumatically into fluid contacting shell 114 to dispel or discharge fresh or used dialysis fluid from pod pump 110.

In either of the pod pump 120 embodiments of FIG. 3 or 4, flexible sheet 112 is (i) pulled towards actuation surface 24 under negative pressure to pull fresh or used dialysis fluid into disposable pumping pod 110, and (ii) pushed away from actuation surface 24 under positive pressure to push fresh or used dialysis fluid from disposable pumping pod 110.

Rigid shells 114 and 120 and flexible plastic sheet 112 of pod pump 110, fluid lines 104, 106a to 106c and 108 and fluid containers 102a to 102c of disposable set 100a may be made of one or more plastic, e.g., polyvinylchloride (“PVC”) or a non-PVC material, such as polyethylene (“PE”), polyurethane (“PU”) or polycarbonate (“PC”). Housing 22a of cycler 20a may be made of any of the above plastics, and/or of metal, e.g., stainless steel, steel and/or aluminum.

FIGS. 1A, 1B and 2 illustrate that cycler 20a includes a control unit 80 having one or more processor 82, one or more memory 84 and a video controller 86 operating with a user interface 88 provided to control each of fluid valves 34, 36a to 36c and 38, each of pneumatic valves 52a, 52b, 54a and 54b, air pump 40, and heater 30 and to receive signals from each of the pressure sensors 48a, 48b, prime sensor/bubble detector 58, and one or more temperature sensor 32 associated with batch or inline heater 30. Control unit 80 may use feedback from one or more temperature sensor 32 to adjust the amount of power delivered to heater 30, e.g., via a proportional, integral, derivative (“PID”) routine, so that heated, fresh dialysis fluid is delivered to the patient at a desired temperature, e.g., 37° C.

User interface 88 may be provided with a touchscreen and/or electromechanical pushbuttons to allow the user or patient to enter parameters for treatment and a display screen for providing information, such as treatment status information. FIG. 1A illustrates user interface 88 off to the side of housing 22a, while FIG. 1B illustrates user interface 88 originally folded into door 26 of housing 22a. Control unit 80 may also include a transceiver (not illustrated) and a wired or wireless connection to a network, e.g., the internet, for sending treatment data to and receiving prescription instructions from a doctor's or clinician's server interfacing with a doctor's or clinician's computer.

In one embodiment, control unit 80 is programmed to cause fresh or used dialysis fluid to be drawn into pod pump 110 using the following procedure. Air pump 40 is configured to be in a negative pressure or suction mode and is placed in pneumatic communication with disposable pod pump 110 via the opening of negative pneumatic valve 52b of pneumatic valve manifold 50. A desired fluid source valve from which fluid is to be drawn into the disposable pumping pod is opened, e.g., dialysis fluid valves 36a to 36c or patient valve 38. Control unit 80 uses pressure feedback from the pod pressure sensor 48 in an algorithm, e.g., a proportional, integral, derivative (“PID”) routine, to regulate air pump 40 to maintain a desired or set negative fluid pressure while fresh or used dialysis fluid is pulled into disposable pumping pod 110. In an embodiment, control unit 80 controls electrical current to air pump 40 to adjust its speed and thus its negative pneumatic pressure output. The desired or set pressure may be different depending on the fluid source, e.g., −1.5 psig to −3.0 psig for pulling from patient P or higher negative pressure for pulling fluid from a dialysis fluid container.

Control unit 80 in one embodiment uses a second algorithm to sense a spike in negative pressure from pod pressure sensor 48a and/or a corresponding drop in the speed of air pump 40 to indicate an end of stroke and that pod pump 110 is filled with fresh or used dialysis fluid, which causes a trigger to stop air pump 40. The speed of air pump 40 is measureable, e.g., via monitoring a moving part of the air pump such as a piston, membrane or shaft rotation speed, and wherein the measured speed is proportional to dialysis fluid filling flowrate. Alternatively or additionally, the speed of air pump 40 may be known or assumed from a speed commanded by control unit 80.

In one embodiment, control unit 80 is programmed to cause fresh or used dialysis fluid to be pumped from pod pump 110 using the following procedure. Air pump 40 is configured to be in a positive pressure mode, which is placed in pneumatic communication with disposable pumping pod 110 via the opening of positive pneumatic valve 52a of pneumatic valve manifold 50. A desired fluid destination valve through which fluid is to be delivered from the disposable pumping pod is opened, e.g., drain valve 34, dialysis fluid heater valves 36a or patient valve 38. Control unit 80 uses pressure feedback from pod pressure sensor 48a in the pressure algorithm, e.g., PID routine, to regulate air pump 40 to maintain a desired or set positive fluid pressure while fluid is discharged from disposable pumping pod 110. Again, control unit 80 may control electrical current to air pump 40 to adjust its speed and thus its positive pneumatic pressure output. The desired or set pressure may be different depending on the fluid destination, e.g., 3.0 psig to 8.0 psig for pushing fresh, heated dialysis fluid to patient P or higher positive pressure for pushing fluid to drain or the heating container 102a.

Control unit 80 in one embodiment uses an additional algorithm to sense a spike in positive pressure from pod pressure sensor 48a and/or a corresponding drop in the speed of air pump 40 to indicate an end of stroke and that pod pump 110 has been emptied of fresh or used dialysis fluid, which causes a trigger to stop air pump 40. The speed of air pump 40 is measureable as discussed above, e.g., via monitoring a moving part of the air pump such as a piston, membrane or shaft rotation speed, and wherein the measured speed is proportional to dialysis fluid emptying flowrate. Alternatively or additionally, the speed of air pump 40 may be known or assumed from a speed commanded by control unit 80.

Control unit 80 is programmed to employ a method 150 for determining how much fresh or used dialysis fluid has been delivered from pod pump 110.

In method 150, control unit 80 measures an initial volume of fresh or used dialysis fluid drawn between flexible sheet 112 and the fluid contacting rigid plastic shell 114 using two sets of pressure measurements and an equation based on the ideal gas law. At oval 152, method 150 begins. At block 154, control unit 80 opens negative pneumatic valve 52b, opens a desired source pinch valve 36a to 36c or 38 and runs air pump 40 so as to create a desired negative pumping pressure and draw fresh or used dialysis fluid into pod pump 110.

At block 156, in a first set of pressure measurements, control unit 80 takes pressure measurements of (i) the air side of the disposable pumping pod 110 using pod pressure sensor 48a and (ii) reference chamber 60 using reference pressure sensor 48b.

At block 158, in a second set of pressure measurements, control unit 80 opens one or more pneumatic reference valve(s) 54a and/or 54b to allow the air side of disposable pumping pod 110 and reference chamber 60 to communicate pneumatically.

Here, both pod pressure sensor 48a and reference pressure sensor 48b measure the pressure of the combined pneumatic cavity. The difference between the first set of measurements at block 156 and the second set of measurements at block 158 is that the first pressure readings at block 156 are taken before pneumatically connecting pumping pod 110 and reference chamber 60, while the second pressure readings at block 158 are taken after pneumatically connecting pumping pod 110 and reference chamber 60.

At block 160, with all values on the right side of the following equation (based on the ideal gas law) known or measured, including the known volume of reference chamber 60, control unit 80 calculates the volume of fresh or used dialysis fluid pulled into disposable pumping pod 110 is as follows:

V_{fluid initial}=V_{reference chamber}*(P_{ref final}−P_{ref initial})/(P_{pump initial}−P_{pump final})

Control unit 80 in method 150 is programmed to measure a final volume of fresh or used dialysis fluid located between flexible sheet 112 and fluid contacting rigid plastic shell 114 using the same two sets of pressure measurements and an equation based on the ideal gas law. At block 162, control unit 80 opens positive pneumatic valve 52a, opens a desired destination pinch valve 34, 36a or 38 and runs air pump 40 so as to create a desired positive pumping pressure and push fresh or used dialysis fluid from pod pump 110.

At block 164, in a first set of pressure measurements, control unit 80 takes pressure measurements of (i) the air side of the disposable pumping pod 110 using the pod pressure sensor 48a and (ii) reference chamber 60 using reference pressure chamber 48b.

At block 166, in a second set of pressure measurements, control unit 80 opens one or more pneumatic reference valve(s) 54a and/or 54b to allow the air side of disposable pumping pod 110 and reference chamber 60 to communicate pneumatically. Here, both pod pressure sensor 48a and reference pressure sensor 48b measure the pressure of the combined pneumatic cavity. The difference between the first set of measurements at block 164 and the second set of measurements at block 166 is that the first pressure readings at block 164 are taken before pneumatically connecting pumping pod 110 and reference chamber 60, while the second pressure readings at block 166 are taken after pneumatically connecting pumping pod 110 and reference chamber 60.

At block 168, with all values on the right side of the following equation (based on the ideal gas law) known or measured, including the known volume of reference chamber 60, control unit 80 calculates the volume of fresh or used dialysis fluid remaining in pod pump 110 after the discharge stroke as follows:

V_{fluid final}=V_{reference chamber}*(P_{ref final}−P_{ref initial})/(P_{pump initial}−P_{pump final})

At block 170, control unit 80 calculates the volume of fresh or used dialysis fluid pumped from pod pump 110 by calculating the difference between the calculated fluid volume after drawing in fluid (V_{fluid initial}) and the calculated fluid volume after pumping fluid out (V_{fluid final}) pumping.

At block 172, control unit adds the volume of fresh or used fluid pumped determined at block 170 with a previously accumulated volume of fresh or used dialysis fluid pumped.

At diamond 174, control unit 80 determines whether the accumulated volume of fresh or used dialysis fluid pumped updated at block 172 meets a required or prescribed volume of fresh or used dialysis fluid to be pumped. If not, then method 150 returns to block 154 and repeats the above sequence. If so, then method 150 ends at oval 176.

The same pumping regime described in connection with method 150 is used to pump fluid from any fluid source described herein to any destination described herein. In steps 158 and 160, both pressure sensors 48a and 48b should read and output the same pressure values to control unit 80. The readings from pressure sensors 48a and 48b could be averaged to provide an average pressure value that is used for operation, or the pressure reading from one of the sensors, e.g., sensor 48a may be used for operation, while the reading from the other pressure sensor 48b may be used as a redundant reading to see if primary pressure sensor 48a has drifted.

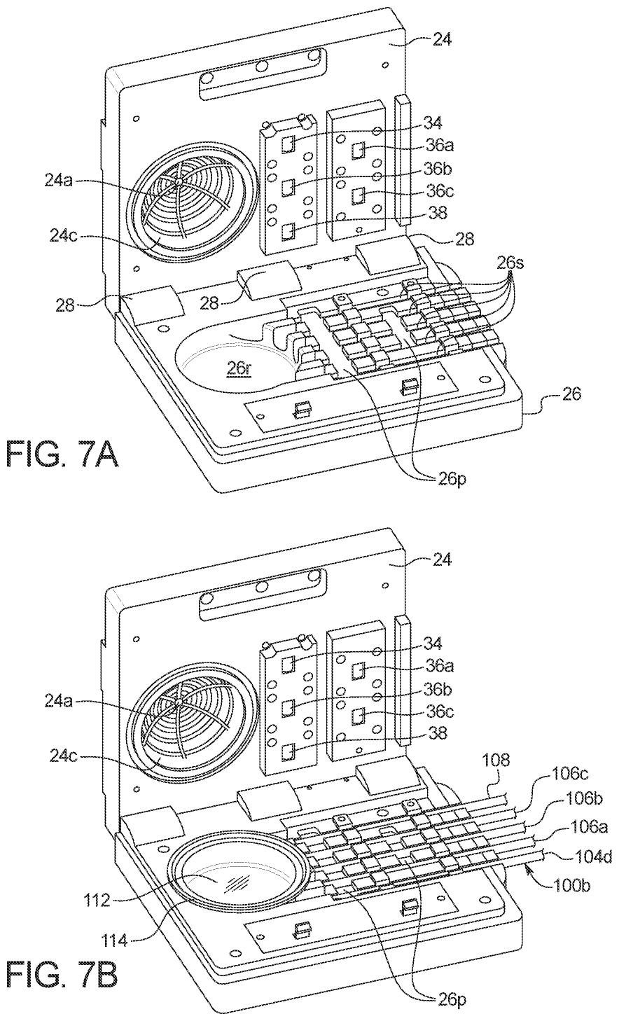

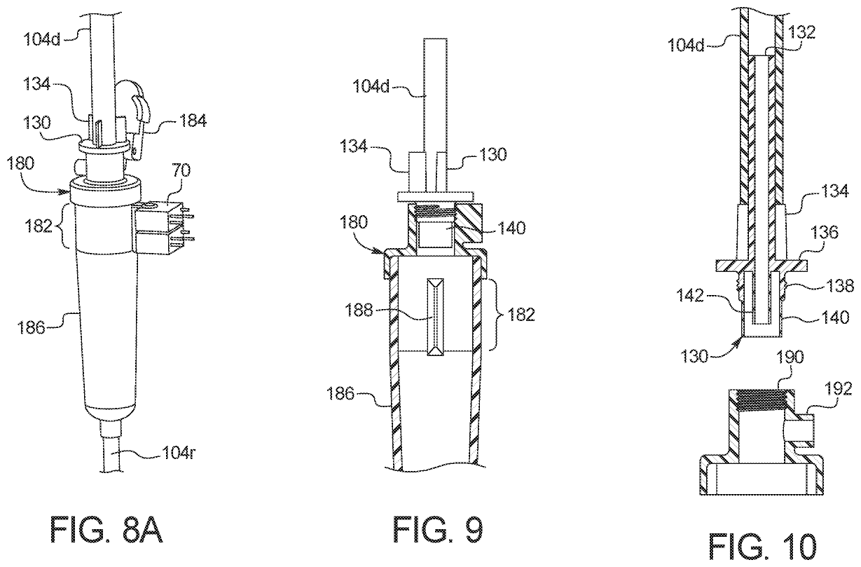

Referring now to FIG. 6, an alternative embodiment of system 10 includes an alternative automated peritoneal dialysis (“APD”) cycler 20b having an alternative housing 22a, which operates with alternative disposable set 100b (FIG. 7B). Disposable set 100b includes pod pump 110, including all structure, functionality and alternatives discussed in connection with disposable set 110a.

As before, a disposable set, here set 100b, including a pod pump 110 is inserted for operation inside of APD cycler 20b, for example, in between an actuation surface and a door 26 of the APD cycler. Door 26, for example, hinges open via one or more hinge located along a bottom of cycler housing 22b, adjacent to actuation surface 24. Disposable set 100b also includes multiple peritoneal dialysis fluid containers or bags 102a to 102c, wherein one of the containers, e.g., container 102a, is placed on top of cycler 20b, which includes a dialysis fluid heater to heat dialysis fluid located originally in container 102a as well as dialysis fluid pumped to container 102a from second and third containers 102b and 102c for subsequent patient fills. As before, the plate or batch heater may be replaced by an inline heater dialysis fluid heater, which heats fresh dialysis fluid as it flows through patient line 108 to the patient.

FIG. 6 also illustrates that disposable set 100b includes each of the five fluid lines, namely, drain line 104d/104r, fresh dialysis fluid container lines 106a to 106c, and patient line 108. Each of lines 104d/104r, 106a to 106c and 108 is fitted into or operates respectively with a pinch valve as discussed above for cycler 20a and disposable set 100a.

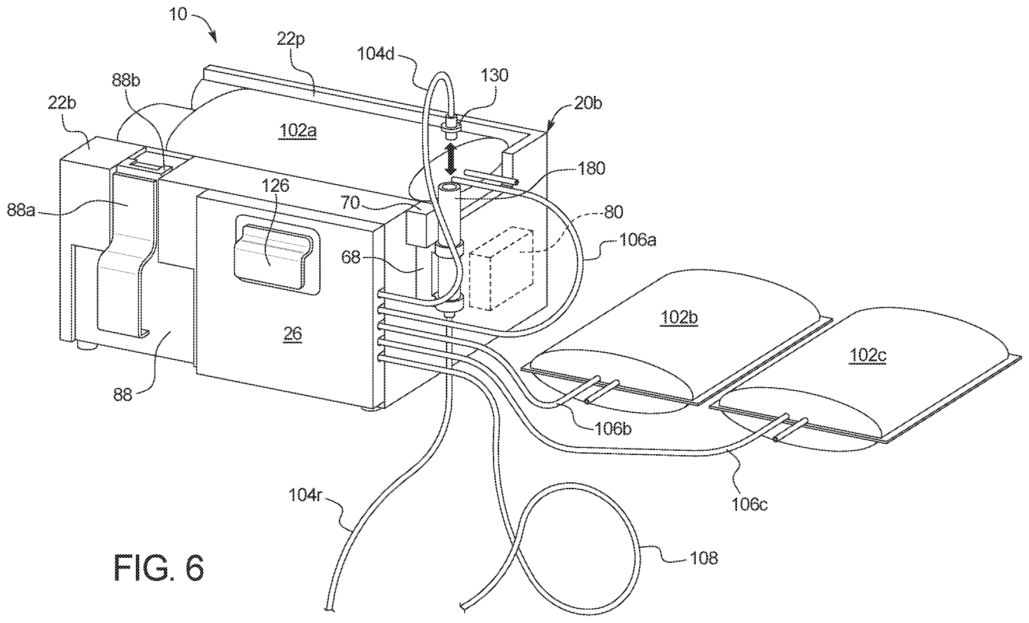

Air pump 40, pneumatic valve manifold 50 and known volume reference chamber 60, including all structure, functionality and alternatives discussed above for cycler 20a are located within housing 22b of cycler 20b. All electronic components of cycler 20b are under control of and/or output to control unit 80 described above. Control unit 80 controls air pump 40 and pneumatic valve manifold 50 using reference chamber 60 to pump fresh and used dialysis fluid to and from pumping pod 110 according to method 150 of FIG. 5 in one embodiment.

One difference with cycler 20b is that user interface 88 is connected to housing 22b via a bracket 88a that connects hingedly to housing 22b via a hinge 88b. When not needed, user interface 88 folds conveniently out of the way and into the front of housing 22b as illustrated in FIG. 6. When needed for setup and treatment, user interface 88 is rotated open and upwardly about hinge 88b in the front of housing 22b where it may be readily viewed.

In the illustrated embodiment of FIG. 6, door 26 is provided with a handle 126, which allows the user to easily open the door to reach disposable set 100b and pumping pod 110. It is contemplated that the closing of door 26 completes an electrical circuit, or that there is a sensor outputting to control unit 80 when door is present, wherein if the electrical circuit is not complete, or if door 26 is not sensed, control unit 80 does not allow the operation of pumping pod 110 and may cause user interface 88 to display an audio, visual or audiovisual alert message.

One difference with disposable set 100b is that the disposable portion of drain line 104d is shortened, reducing waste and cost. Disposable portion of drain line 104d includes a connector 130 that attaches to a reusable drip chamber 180 illustrated and described in more detail below. A reusable portion of drain line 104r extends from the bottom of reusable drip chamber 180 to a drain container or bag or to a house drain, such as a toilet, bath or sink. Reusable portion of drain line 104r is significantly longer than disposable portion 104d, reducing cost. FIG. 6 illustrates that drip chamber 180 is held in a clip 68 attached to housing 22b. A sensor 70 (or a pair of sensors 70) outputting to control unit 80 is/are provided with clip 68 and is/are located so as to sense a desired area of reusable drip chamber 180 when inserted into clip 68.

FIG. 6 further illustrates that container or bag 102a may be placed in a heater pan 22p, which holds heater 30 and temperature sensors 32 and cradles a portion of the sides of container or bag 102a, holding the container steady and tending to insulate the container. It should be appreciated that while system 10 of FIGS. 1 and 6 is illustrated having three dialysis fluid containers or bags 102a, 102b and 102c, system 10 may be provided alternatively with only two containers or more than three dialysis fluid containers.

Referring now to FIGS. 7A and 7B, a section of cycler 20b of system 10 is illustrated showing door 26 rotated open for both situations in which disposable set 100b is unloaded (FIG. 7A) and loaded (FIG. 7B). FIGS. 7A and 7B illustrate that the opened door 26 exposes actuation surface 24 and pinch valves 34, 36a to 36c and 38 located along actuation surface 24. Actuation surface 24 also includes or defines aperture or port 24a, which in the illustrated embodiment is provided in the middle of a hemispherical cavity 24c, which accepts diaphragm or membrane 112 (FIG. 3) when placed under negative pneumatic pressure via aperture or port 24a. Hemispherical cavity 24c alternatively accepts pneumatic rigid plastic shell 120 (FIG. 4) having central port 122 for sealed insertion into aperture or port 24a.

FIG. 7A illustrates that the inside of door 26 includes or defines a pump recess 26r for receiving fluid contacting rigid plastic shell 114 (FIGS. 3 and 4) of pod pump 110. The inside of door 26 also includes or defines line or tube receiving slots 26s for receiving lines or tubes 104d/104r, 106a to 106c and 108. The inside of door 26 also includes or defines press plates 26p against which pinch valves 34, 36a to 36c and 38 pinch or occlude lines or tubes 104d/104r, 106a to 106c and 108.

FIG. 7B illustrates that disposable set 100b is easily loaded into pump recess 26r and line or tube receiving slots 26s. Rigid plastic shell 114 of pod pump 110 is held firmly within pump recess 26r, while lines or tubes 104d/104r, 106a to 106c and 108 are organized and held straight within line or tube receiving slots 26s, for example, so that they are oriented properly for actuation via pinch valves 34, 36a to 36c and 38. Pod pump 110 and lines or tubes 104d/104r, 106a to 106c and 108 are also held in place such that door 26 may be rotated up about hinges 28 and locked against actuation surface 24 for treatment. Setup for the patient or caregiver is accordingly relatively simple. In addition to loading pod pump 110 and lines or tubes 104d/104r, 106a to 106c and 108 into door 26 and closing the door, the patient or caregiver also loads container 102a into heater pan 22p, locates a distal end of patient line 108 for priming, and connects connector 130 at the disposable portion of drain line 104d to the top of reusable drip chamber 180.

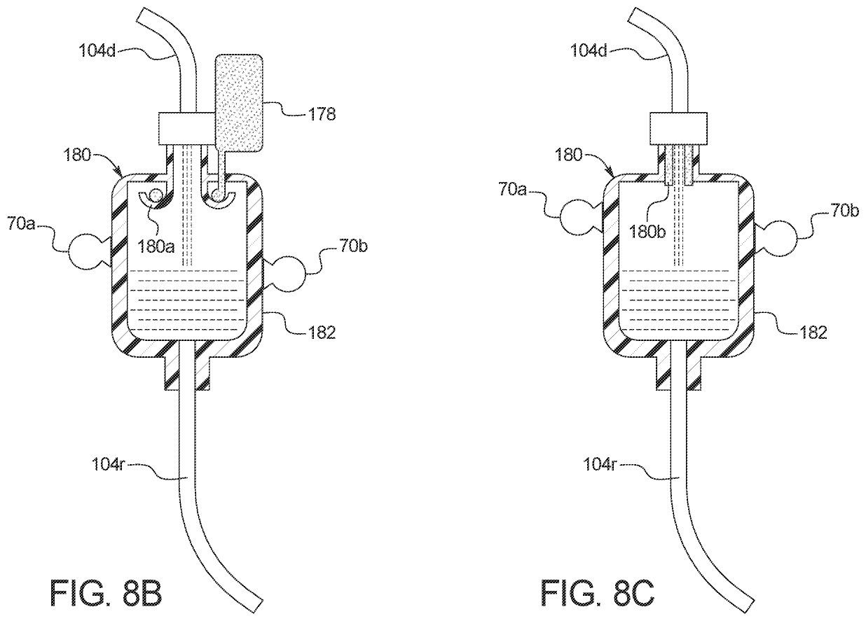

Referring now to FIGS. 8A to 10, reusable drip chamber 180, sensor(s) 70 and connector 130 are illustrated in more detail. FIG. 8A illustrates that sensor(s) 70 (attached to housing 22b of cycler 20b in FIG. 6) is/are located such that when reusable drip chamber 180 is mounted in clip 68, a sensing portion 182 of reusable drip chamber 180 is aligned with sensor(s) 70. Sensor(s) 70 is in various implementations an optical sensor, a capacitance sensor, an ultrasonic sensor, a proximity sensor, a magnetic sensor and combinations thereof. Sensor(s) 70 outputs to control unit 80, which is programmed to halt a drain immediately if the reading from sensor(s) 70 is not as expected, does not meet a threshold or does not fall inside of an acceptable range.

Sensor(s) 70 is/are provided to look to ensure that an air column exists within sensing portion 182 of reusable drip chamber 180. The air column helps to ensure the sterility of used dialysis fluid flowing through disposable portion of drain line 104d. That is, over time impurities may form in reusable portion of drain line 104r. The air column formed in drip chamber 180, however, ensures that there is no fluid path for any pathogens to flow from reusable portion of drain line 104r to disposable portion of drain line 104d. The only path is for the pathogens to migrate up the inner wall of sensing portion 182, which is a tortuous path. Additionally, the small disposable portion of drain line 104d still provides a relatively long path for any pathogen(s) that is/are able to migrate into disposable portion 104d, against the flow of used dialysis fluid and during a single treatment.

FIG. 8A illustrates that reusable drip chamber 180 may include a hinged cap 184, e.g., via a living hinge formed in the same mold with the rest of drip chamber 180. Drip chamber 180 may be molded in one or more pieces using any of the materials discussed herein and may be clear (e.g., for effluent evaluation, or opaque for operation with an optical sensor). Alternatively, cap 184 may be a separate piece that is hingedly connected to drip chamber 180. In any case, the patient or caregiver replaces cap 184 onto reusable drip chamber 180 when disposable portion of drain line 104d and connector 130 are removed from the drip chamber.

FIG. 8A also illustrates that reusable drip chamber 180 includes an elongated body 186, which may be slightly tapered so as have a smaller diameter at its bottom. The used dialysis fluid or effluent falls from the exit port of connector 130 into a pool or column of used dialysis fluid or effluent formed in elongated body 186. If the effluent level rises too much and extends into sensing area 182, sensor(s) 70 detects same and control unit 80 responds by halting the current drain.

FIGS. 8A and 9 illustrate connector 130 removably and sealingly connected, e.g., threaded, to reusable drip chamber 180. The threaded connection may be any suitable threaded connection, e.g., a luer connection. Alternatively or additionally, the removably sealed connection may be via an o-ring provided by reusable drip chamber 180.

FIGS. 8B and 8C illustrate alternative embodiments for reusable drip chamber 180. FIGS. 8B and 8C both illustrate that sensors 70a and 70b may be located on different sides of sensing portion 182. FIGS. 8A to 8C each illustrate that multiple sensors 70 (e.g., sensors 70a and 70b) may be located at different elevations, providing varied alarm or alert levels to control unit 80. In FIGS. 8B and 8C, for example, sensor 70b is located beneath sensor 70a, so that a detection of a liquid level (dotted lines in FIGS. 8B and 8C) from sensor 70b is not as critical as a liquid level detection from sensor 70a. In an embodiment, control unit 80 may be programmed to take an evasive action upon receiving a liquid level detection from sensor 70b, e.g., slow or stop an ongoing drain, which may or may not be accompanied by a corresponding alert or alarm at user interface 88. A more critical response occurs when control unit 80 receives a liquid level detection from sensor 70a, e.g., stopping an ongoing drain and alerting or alarming at user interface 88 that treatment is temporarily halted. The patient may also be prompted to look for a kink or occlusion in reusable portion of drain line 104r.

FIG. 8B also illustrates that the inlet of reusable drip chamber 180 may be provided, e.g., molded, with a disinfectant well 180a, which receives disinfectant, e.g., Betadine, from a disinfectant source or reservoir 178. The disinfectant from well 180a coats the inner walls of sensing portion 182 to prevent pathogen migration and may also mix with the patient's effluent if the effluent level rises so as to reach disinfectant well 180a. Here, pathogens in the patient's effluent are destroyed via the mixed disinfectant.

FIG. 8C further illustrates that the inlet of reusable drip chamber 180 may be provided, e.g., press-fitted and/or adhered to, with a disinfectant sponge collar 180b, which is infused with disinfectant, e.g., Betadine. Disinfectant sponge collar 180b, e.g., a cylindrical collar, may alternatively be provided, e.g., press-fitted and/or adhered to, as part of the distal connector 130 of the disposable portion of drain line 104d. In either case, the disinfectant from sponge collar 180b prevents pathogen migration and may also mix with the patient's effluent if the effluent level rises so as to reach disinfectant sponge collar 180b.

Here again, pathogens in the patient's effluent are destroyed via the mixed disinfectant.

FIG. 9 also illustrates that sensing portion 182 may include a window or slot 188 that operates, e.g., abuts against or connects to sensor(s) 70. Window or slot 188 is provided for operation with an optical version of sensor(s) 70, wherein sensing portion 182 is opaque to outside light except at window or slot 188 for allowing optical sensor(s) 70 to “see” into sensing portion 182 and look for the air column. The optical version of sensor(s) 70 may include an emitter and a receiver, wherein the amount of light received at the receiver is different if sensing portion 182 is, or is partially, filled with used dialysis fluid instead of being filled with air.

FIG. 10 illustrates connector 130 in detail. Connector 130 may be made, e.g., molded, from any of the materials discussed herein. Connector 130 in the illustrated embodiment includes a tube 132, e.g., rigid tube, which is sized to press-fit into the distal end of disposable portion of drain line 104d. A stop 134 is provided to mark an end of the insertion of tube 132 into disposable portion of drain line 104d. Tube 132 extends to a flange 136 that enables the user to manipulate connector 130 and disposable portion of drain line 104d for connection to reusable drip chamber 180.

In the illustrated embodiment, male threads 138 are provided on the opposite side of flange 136 from stop 134. Male threads 138 mate sealingly with female threads 190 provided at the top of reusable drip chamber 180. A shroud 140 extends from male threads 138 past a concentric inner port 142 from which used dialysis fluid forms droplets that fall from connector 130 into reusable drip chamber 180. The additional extension of shroud 140 past inner port 142 tends to prevent a user from touching an end of inner port 142 and potentially contaminating same.

When connector 130 is inserted, e.g., threaded, into the top of reusable drip chamber 180, shroud 140 extends past a port 192 of the drip chamber. Port is provided for connection to an air or pneumatic line leading to an air pump, which may be system air pump 40 or a separate small, dedicated air pump under control of control unit 80. For example, the air or pneumatic line may extend from port 192, through an aperture formed in housing 22a to 22c of cyclers 20a to 20c, to the air pump. The air pump pumps air into reusable drip chamber 180 to help ensure that the air column is present. Air may be pumped into reusable drip chamber 180 continuously during drain or upon sensor(s) 70, e.g., lower sensor 70b in FIGS. 8B and 8C, sensing a certain level of used dialysis fluid building in sensing portion 182. The air pump may also be activated at the end of a patient drain phase to blow out reusable portion of drain line 104r with the goal of preventing pathogen growth and migration along the reusable drain line.

In an alternative embodiment, the air or pneumatic line extends from port 192 to a hydrophobic vent located at its distal end, which may be routed so as to reside within housing 22a to 22c. The hydrophobic vent filters air entering the line due to negative pressure created via a venturi effect of the effluent fluid flowing through reusable drip chamber 180. The air pulled in through port 192 extends to sensing portion 182 to help maintain the air column.

FIG. 6 illustrates reusable drip chamber 180 operating with disposable set 100b of cycler 20b, which includes pod pump 110 having a fluid contacting fluid contacting rigid plastic shell 114 to which flexible sheet 112 is attached. After treatment, disposable set 100b including disposable portion of drain line 104d is removed and discarded, while reusable portion of drain line 104r is retained. The distal end of reusable portion 104r may be capped after treatment, e.g., via a tethered cap provided at the distal end or via a blind port at cycler 20b, wherein cycler 20b may provide a sensor (of any of the types described herein for sensor(s) 70) to confirm that the distal end of reusable portion 104r has been capped. Likewise, the top of reusable drip chamber 180 is capped after treatment, e.g., via a tethered cap provided at the top of reusable drip chamber 180. In an alternative embodiment, the patient or user does not remove disposable portion of drain line 104d after treatment but instead leaves the line in place until the next treatment. In this manner, the top of reusable drip chamber 180 remains capped and sealed from the environment until the next treatment. Here, the patient or user just needs to cap the distal end of reusable portion 104r after treatment in any of the manners described herein.

It should be appreciated however that reusable drip chamber 180 and drain line portions 104d and 104r do not have to be provided with or operate with a disposable set, with a cycler that operates a disposable set, or with a pneumatically operated cycler. Reusable drip chamber 180 and drain line portions 104d and 104r may operate alternatively with any type of pumping, such as peristaltic pumping, piston pumping, centrifugal pumping, etc. The pumping (valving and heating) may further alternatively be durable, not disposable, in which fresh and used PD fluid flows through the durable PD fluid pump over many treatments. The durable cycler may be disinfected in between treatments. Here, disposable portion of drain line 104d may be plugged directly into the cycler during treatment to communicate fluidly with one or more internal, reusable drain line.

For disinfection, the patient or user may remove disposable portion of drain line 104d from a drain connector of the cycler. If so, the drain connector includes a cover or cap that seals the drain connector closed during disinfection. The patient or user may remove the disposable portion of drain line 104d from reusable drip chamber 180 so that portion 104d can be discarded. The patient or user caps reusable drip chamber 180 and the distal end of reusable portion 104r as discussed above. Alternatively, the patient or user leaves the disposable portion of drain line 104d in place during disinfection, connected to the drain connector of the cycler and the top of reusable drip chamber 180. Here, the cycler fluidly isolates the drain connector, or at least a portion thereof, during disinfection, so that disinfection fluid does not flow into the left-in-place disposable portion of drain line 104d. In any of the above embodiments, disinfection may then proceed assuming any other needed disinfection connections (e.g., patient line, dialysis fluid supply lines) have been made.

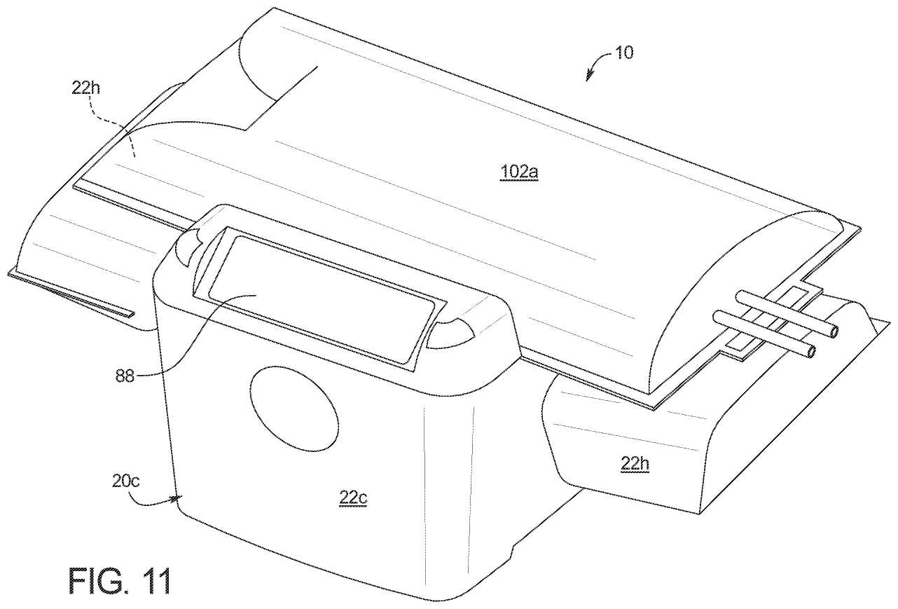

FIG. 11 illustrates a further alternative cycler 20c for system 10 having an alternative housing 22c. Alternative cycler 20c includes control unit 80 controlling air pump 40 and pneumatic valve manifold 50 using reference chamber 60 to pump fresh and used dialysis fluid to and from pumping pod 110 according to method 150 of FIG. 5 in one embodiment. Alternative cycler 20c may operate with either disposable set 100a or 100b.

The primary differences with alternative cycler 20c are that user interface 88 is built into or integrated with housing 22c instead of being hinged to the housing. User interface may likewise be built into housings 22a and 22b of cyclers 20a and 20b. A primary difference with alternative cycler 20c is that the lid of housing 22c may be split into two halves 22h, e.g., equally sized halves. Each half 22h may then hinge outwardly open for receiving and supporting a portion of container or bag 102a. When halves 22h are open, a heating tray having heater 30 and temperature sensors 32 under control of control unit 80 is exposed for controllably heating fresh dialysis fluid located within container or bag 102a. Rotatable halves 22h enable the overall size of housing 22c to be smaller and to not be constrained in two dimensions to being at least the size of container or bag 102a.

Referring now to FIG. 12, an embodiment for pinch valves 34, 36a to 36c and 38 useable with system 10 is illustrated. In the illustrated embodiment, pinch valves 34, 36a to 36c and 38 include a housing 90, which is bolted to an electrically actuated linear actuator 92 operated under control of control unit 80. In the illustrated embodiment, four bolts or screws pass through respective clearance apertures 90a to 90d formed in housing 90 and thread into respective threaded holes or bores 92a to 92d provided in linear actuator 92. To mount the pinch valves, housing 90 may be mounted to actuation surface 24, or linear actuator 92 may be mounted to some portion of housing 22a to 22c of cyclers 20a to 20c, respectively.

Linear actuator 92 in one embodiment includes a rotary nut motor having female threads (not illustrated) that rotate threadingly around male threads of a threaded shaft 94. The female threads rotate but are constrained so as not to translate along the axis of threaded shaft 94, which causes the threaded shaft to translate back and forth along the central axis extending through shaft 94 and linear actuator 92. Linear actuator 92 is bidirectional so that when control unit 80 causes the female threads to turn in a first direction, threaded shaft 94 is pulled into the linear actuator, opening a line 104, 106a to 106c or 108. When control unit 80 instead causes the female threads to turn in a second direction, threaded shaft 94 is pushed out of the linear actuator, closing a line 104, 106a to 106c or 108.

A mounting flange 96 is located about threaded shaft 94. Mounting flange 96 includes or defines clearance holes 96a to 96d that accept screws that thread into respective threaded holes or bores 98a to 98d provided in a plunger 98. Plunger 98 is thus fixed to mounting flange 96. Plunger 98 and mounting flange 96 constrain a compression spring 194. When plunger 98 is pulled away from and is not contacting a line 104, 106a to 106c or 108, compression spring 194 pushes plunger 98 against a collar 196, which is fixed to threaded shaft 94, e.g., via a set screw, which in turn pulls mounting flange 96 to abut against or away from a collar 196. When plunger 98 is pushed towards and makes contact with a line 104, 106a to 106c or 108, compression spring 194 compresses and allows plunger 98 and mounting flange 96 to translate towards linear actuator 92 slightly relative to fixed collar 196 until the compression spring 194 becomes fully compressed. The slight amount of play provides greater tolerance to the pinch valve 34, 36a to 36c and 38.

The distal end of plunger 98 extends through an aperture 90e provided in housing 90 to reach line 104, 106a to 106c or 108. Aperture 90e also centers and constrains the movement of the distal end of plunger 98. In one embodiment, the combination of the movement of threaded shaft 94 and the force of compression spring 194 (sized selectively in at least one of length, coil diameter and spring material) provides enough extension distance and spring force to fully occlude lines 104, 106a to 106c and 108 even under the highest possible positive dialysis fluid pressure, e.g., 8 psig.

In the illustrated embodiment, pinch valve 34, 36a to 36c or 38 is provided with a sensor 198, which may be attached to the back of linear actuator 92 or to a portion of housing 22a to 22c of cyclers 20a to 20c, respectively. In either case, sensor 198 is positioned to sense whether a proximal end 94p of threaded shaft 94 is present or not, that is, whether the proximal end 94p has been pulled back to the point that it extends through the back end of linear actuator 92 (indicating an open or fluid flow valve position). Sensor 198 may be an optical, capacitive, ultrasonic or other type of sensor outputting to control unit 80, which is capable of detecting the presence or not of the proximal end 94p of threaded shaft 94. In an embodiment, control unit 80 is programmed to look to see from the output of sensor 198 that the proximal end 94p is not present at a time when pinch valve 34, 36a to 36c or 38 is supposed to be closed to occlude flow through line 104, 106a to 106c and 108. If the output from sensor 198 indicates that proximal end 94p is not present at a time when the pinch valve is supposed to be closed, then treatment proceeds as planned. If the output from sensor 198 indicates that proximal end 94p is present at a time when the pinch valve is supposed to be closed, then treatment is stopped and an audio, visual or audiovisual alarm is provided at user interface 88 in one embodiment.

Each of the components of pinch valve 34, 36a to 36c or 38 described above, including housing 90, linear actuator 92, threaded shaft 94, mounting flange 96, plunger 98, spring 194, collar 196, and sensor 198 may be formed from metal, such as stainless steel, steel and/or aluminum, and/or molded from plastic, such as polyvinylchloride (“PVC”) or a non-PVC material, such as polyethylene (“PE”), polyurethane (“PU”) or polycarbonate (“PC”).

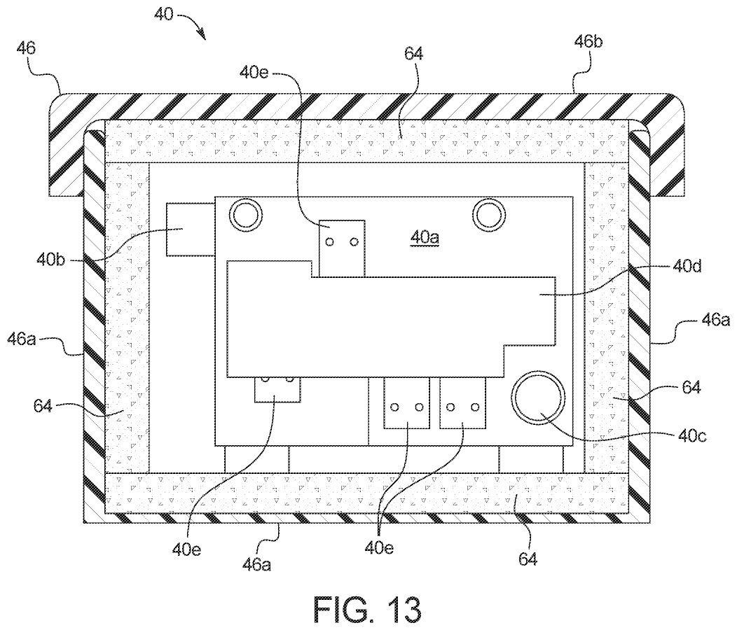

Referring now to FIG. 13, an embodiment for air pump 40 is illustrated, which is useable with any of cyclers 20a to 20c of system 10. Air pump 40 in the illustrated embodiment includes a pump body 40a. Pump body 40a includes a compressor that creates positive pneumatic pressure for the pod pump discharge strokes described herein and negative pneumatic pressure for the pod pump draw strokes described herein. In an embodiment, the positive and negative pressures are delivered via a single air tube or line to pod pump 110. Pump body 40a includes vents 40b and 40c that allow air into and out of the compressor. Air pump 40 may also include its own microcontroller board 40d that operates with control unit 80, and a plurality of electrically actuated pneumatic solenoid valves 40e controlled via microcontroller board 40d under the command of control unit 80.

An issue with air pumps in general is noise, which is heightened in the present disclosure because a primary mode of use for cyclers 20a to 20c is at night while the patient sleeps. Air pump 40 of the present disclosure mitigates audible noise via a box-in-a-box configuration.

In the illustrated embodiment, pump body 40a is housed inside of a first noise-reducing enclosure, which is formed from a plurality of acoustic foam sheets 64.

Acoustic foam sheets 64 may for example be six to fifteen millimeters thick or even thicker, e.g., up to twenty-five millimeters thick and may be made of high density polyethylene (“HDPE”). Acoustic foam sheets 64 may be secured together to form the first enclosure, e.g., adhered and/or fastened together, with the top acoustic foam sheet 64 left unattached so that pump body 40a may be placed inside of the first enclosure. Acoustic foam sheets 64 are alternatively secured, e.g., adhered and/or fastened, to the insides of walls 46a and 46b of a second or outer noise-reducing enclosure. Walls 46a and 46b may be made of any of the metal or plastic materials discussed herein and may be four to twelve millimeters thick. Walls 46a include or form the sides and bottom of the outer enclosure, while wall 46b includes the top of the outer enclosure, which is removable so that pump body 40a may be placed inside of the first and second enclosures. The first and second enclosures may include mating one or more aperture(s) that allow pneumatic tubing or lines and electrical wiring to run to pump body 40a.

The outer enclosure is also made of a noise reducing or attenuating material. The combination of the inner and outer enclosures allows the audible noise output from air pump 40 to be reduced to about thirty-one to thirty-two dB. It is contemplated to further reduce the noise output by placing spacers between the inner enclosure (formed from acoustic form sheets 64) and the outer enclosure (formed from walls 46a and 46b) so as to create an air gap between the enclosures. The air gap acts as a third noise reducing layer.

It should be understood that various changes and modifications to the presently preferred embodiments described herein will be apparent to those skilled in the art. For example, pneumatic valves may be used instead of electrically actuated pinch valves. In another example, separate positive and negative air pumps may be used to supply positive and negative air pressure instead of single air pump 40. In a further example, heater 30 could be located remote from cycler 20a. Such changes and modifications can be made without departing from the spirit and scope of the present subject matter and without diminishing its intended advantages. It is therefore intended that such changes and modifications be covered by the appended claims.

The invention is claimed as follows:

1. A peritoneal dialysis system comprising:
a cycler including
a dialysis fluid pump,
a plurality of valve actuators, and
a control unit configured to control the dialysis fluid pump and the plurality of valve actuators; and
a drain line including
a disposable portion,
a reusable portion, and
a drip chamber located between the disposable portion and the reusable portion, the drip chamber configured to create an air column that dissuades pathogen migration from the reusable portion to the disposable portion.

2. The peritoneal dialysis system of claim 1, wherein the drip chamber is reusable and provided with the reusable portion of the drain line.

3. The peritoneal dialysis system of claim 1, wherein the dialysis fluid pump includes a pneumatic valve manifold and an air pump positioned and arranged to supply pneumatic pressure to the pneumatic valve manifold.

4. The peritoneal dialysis system of claim 3, wherein the disposable portion of the drain line is provided as part of a disposable set including a pump actuation portion operable with the dialysis fluid pump, and a valve actuation portion operable with the plurality of valve actuators.

5. The peritoneal dialysis system of claim 4, wherein the pump actuation portion of the disposable set includes a pod pump having a flexible sheet, one side of the flexible sheet positioned and arranged during operation to receive pneumatic pressure via the air pump and pneumatic valve manifold.

6. The peritoneal dialysis system of claim 4, wherein the plurality of valve actuators include pinch valve actuators.

7. The peritoneal dialysis system of claim 6, wherein the valve actuation portion of the disposable set includes a plurality of lines, including the drain line, positioned and arranged to be actuated by the pinch valve actuators

8. The peritoneal dialysis system of claim 3, which includes a pneumatic pressure sensor positioned and arranged to detect pneumatic pressure, and a control unit configured to use an output of the pneumatic pressure sensor as feedback to adjust the air pump according to a set pneumatic pressure.

9. The peritoneal dialysis system of claim 1, wherein the reusable portion of the drain line is longer than the disposable portion of the drain line.

10. The peritoneal dialysis system of claim 1, which includes a connector located at an end of the disposable portion of the drain line, the connector configured to connect to the drip chamber, the connector configured to dissuade touch contamination.

11. The peritoneal dialysis system of claim 1, wherein the cycler includes a clip for holding the drip chamber.

12. The peritoneal dialysis system of claim 1, wherein the cycler includes a sensor outputting to the control unit, the drip chamber positioned and arranged to enable the sensor to sense the air column.

13. The peritoneal dialysis system of claim 12, wherein the sensor is an optical sensor, an ultrasonic sensor, a capacitance sensor, a proximity sensor, a magnetic sensor or combinations thereof.

14. The peritoneal dialysis system of claim 12, wherein the sensor incudes first and second sensors spaced elevationally apart, the control unit programmed to react differently to the first sensor versus the second sensor detecting an interruption of the air column.

15. The peritoneal dialysis system of claim 12, wherein the drip chamber includes a window or slot for operation with the sensor.

16. The peritoneal dialysis system of claim 1, wherein the drip chamber includes a well configured to receive and hold disinfectant.

17. The peritoneal dialysis system of claim 16, which includes a disinfectant reservoir positioned and arranged to deliver disinfectant to the well.

18. The peritoneal dialysis system of claim 1, which includes a disinfectant sponge configured for insertion into the drip chamber.

19. The peritoneal dialysis system of claim 18, wherein the disinfectant sponge is provided as part of the disposable portion of the drain line.

20. The peritoneal dialysis system of claim 1, wherein the drip chamber includes a port connectable to a pneumatic line for receiving air to help maintain the air column.

21. The peritoneal dialysis system of claim 20, which includes an air pump for delivering air through the pneumatic line or a hydrophobic vent placed at a distal end of the pneumatic line enabling air to be drawn into the drip chamber under negative pressure.

22. The peritoneal dialysis system of claim 1, which includes a sealing mechanism for sealing a distal end of the disposable portion of the drain line after treatment.

23. The peritoneal dialysis system of claim 22, wherein the sealing mechanism includes a tethered cap or a blind port provided at the cycler.

24. The peritoneal dialysis system of claim 1, which (i) includes a sealing mechanism for sealing the drip chamber after treatment or (ii) is configured such that the disposable portion of the drain line remains connected to the drip chamber after treatment.
